SEQUENCE LISTING (10 sequences)

SEQ ID NO: 1 - DNA, 17 bp, Homo Sapiens
gcaccacgttcccgtgg

SEQ ID NO: 2 - DNA, 20 bp, Homo Sapiens
cgttcccgtggttcccgtgg

SEQ ID NO: 3 - DNA, 17 bp, Homo Sapiens
tccacccgttcccgtgg

SEQ ID NO: 4 - DNA, 19 bp, Homo Sapiens
cccgtggcggttcccgtgg

SEQ ID NO: 5 - DNA, 19 bp, Homo sapiens
ttagtatataggactaaca

SEQ ID NO: 6 - DNA, 18 bp, Homo sapiens
gaggattggagaggtagc

SEQ ID NO: 7 - DNA, 23 bp, Homo sapiens
ttggaatcccaggtgttgttctc

SEQ ID NO: 8 - DNA, 18 bp, Homo sapiens
gtaggtaatcttcaggct

SEQ ID NO: 9 - DNA, 18 bp, Homo sapiens
aggctaattgattttgac

SEQ ID NO: 10 - DNA, 19 bp, Homo sapiens
tcttaggaagcgaagcaat

(19) United States
(12) Patent Application Publication
(10) Pub. No.: US 2022/0316006 A1
(43) Pub. Date: Oct. 6, 2022

(54) PLASMA AND CEREBROSPINAL FLUID MIRNA BIOMARKERS IN INTRACEREBRAL AND SUBARACHNOID HEMORRHAGE

(71) Applicant: OCHSNER HEALTH SYSTEM, New Orleans, LA (US)

(72) Inventors: Ifeanyi IWUCHUKWU, New Orleans, LA (US); Doan NGUYEN, New Orleans, LA (US)

(73) Assignee: OCHSNER HEALTH SYSTEM, New Orleans, LA (US)

(21) Appl. No.: 17/286,411

(22) PCT Filed: Oct. 16, 2019
(86) PCT No.: PCT/US19/56464

§ 371 Date: Apr. 16, 2021

Related U.S. Application Data
(60) Provisional application No. 62/747,041, filed on Oct. 17, 2018.

(51) Int. Cl. C12Q 1/6876

(52) U.S. Cl. CPC C12Q 1/6876; C12Q 2600/178; C12Q 2600/158; C12Q 2600/112; C12Q 1/6837

(57) ABSTRACT

Methods of detecting, treating and/or preventing subarachnoid hemorrhage (SAH) and intracerebral hemorrhage (ICH) or an associated inflammation response in a subject. Compositions for detecting, treating and/or preventing subarachnoid hemorrhage (SAH) and intracerebral hemorrhage (ICH) or an associated inflammation response in a subject.

### CROSS-REFERENCE TO RELATED APPLICATION

The present application is a national phase entry under 35 U.S.C. § 371 of International Application No. PCT/US2019/056464, filed Oct. 16, 2019, which designates the United States of America, which claims the priority benefit of the earlier filing of U.S. Provisional Application No. 62/747,041, filed Oct. 17, 2018, which are specifically incorporated herein by reference in their entireties and for all purposes.

### TECHNICAL FIELD

This disclosure relates to methods and compositions for detecting, diagnosing, and treating brain hemorrhage, such as subarachnoid hemorrhage (SAH) and intracerebral hemorrhage (ICH), in order to prevent, anticipate, and better treat a brain hemorrhage, including hemorrhagic stroke. Specifically, newly discovered biomarkers, and novel methods for using them, are described.

### BACKGROUND

A stroke occurs when the flow of blood to part of the brain is cut off or significantly reduced. Without the oxygen carried by the blood, brain cells can die quickly, which can cause permanent brain damage. Strokes can be major or minor and the consequences can range from complete recovery to fatality. There are two types of strokes: ischemic and hemorrhagic. An ischemic stroke is caused by lack of blood flow to brain tissue. This can happen when the arteries in the brain narrow due to a condition such as atherosclerosis. In contrast to an ischemic stroke a hemorrhagic stroke occurs when an artery in the brain bursts and causes localized bleeding in the surrounding tissues. This discharge of blood can disrupt the normal circulation to the brain leading to stroke. The damage a hemorrhage wreaks on the brain is determined by the size of the hemorrhage, the amount of swelling in the skull and how quickly the bleeding is controlled. Hemorrhagic strokes account for about 13% of strokes. Methods are needed for detecting hemorrhagic stroke as well as discriminating between types of hemorrhagic stroke. The present disclosure meets those needs.

### Nucleic Acid Sequences

The nucleic acid sequences disclosed herein are shown using standard letter abbreviations for nucleotide bases. Only one strand of each nucleic acid sequence is shown, but the complementary strand is understood as included by any reference to the displayed strand.

PC-5p-585_8337:
(SEQ ID NO: 1)
GCACCACGTTCCCGTGG.
PC-5p-218_26568:
(SEQ ID NO: 2)
CGTTCCCGTGGTTCCCGTGG.
PC-5p-1115_3460:
(SEQ ID NO: 3)
TCCACCCGTTCCCGTGG.
PC-5p-445_12007:
(SEQ ID NO: 4)
CCCGTGGCGGTTCCCGTGG.
PC-3P-7630_279:
(SEQ ID NO: 5)
TTAGTATATAGGACTAACA
PC-5P-1731_1950:
(SEQ ID NO: 6)
GAGGATTGGAGAGGTAGC
PC-3p-4244_570:
(SEQ ID NO: 7)
TTGGAATCCCAGGTGTTGTTCTC
PC-3p-3144_857:
(SEQ ID NO: 8)
GTAGGTAATCTTCAGGCT
PC-5p-6497_338:
(SEQ ID NO: 9)
AGGCTAATTGATTTTGAC
PC-5p-3305_806:
(SEQ ID NO: 10)
TCTTAGGAAGCGAAGCAAT

### BRIEF DESCRIPTION OF THE DRAWINGS

Embodiments will be readily understood by the following detailed description in conjunction with the accompanying drawings and the appended claims. Embodiments are illustrated by way of example and not by way of limitation in the figures of the accompanying drawings.

FIG. 1 is two heat maps showing differential expression of microRNAs (miRNAs) in cerebrospinal fluid (CSF) and plasma, respectively.

### DETAILED DESCRIPTION OF DISCLOSED EMBODIMENTS

### Terms

In the following detailed description, reference is made to the accompanying drawings that form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

Unless otherwise noted, technical terms are used according to conventional usage. Definitions of common terms in molecular biology can be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710); and other similar references.

Suitable methods and materials for the practice or testing of this disclosure are described below. Such methods and materials are illustrative only and are not intended to be limiting. Other methods and materials similar or equivalent to those described herein can be used. For example, conventional methods well known in the art to which this disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999. In addition, the materials, methods, and examples are illustrative only and not intended to be limiting.

To facilitate review of the various embodiments of the disclosure, the following explanations of specific terms are provided:

Administration: To provide or give a subject an agent, such as a therapeutic agent, by any effective route. Exemplary routes of administration include, but are not limited to, injection (such as subcutaneous, intramuscular, intradermal, intraperitoneal, and intravenous), oral, intraductal, sublingual, rectal, transdermal, intranasal, and inhalation routes.

Agent: Any protein, nucleic acid molecule (including chemically modified nucleic acids), compound, small molecule, organic compound, inorganic compound, or other molecule of interest. Agent can include a therapeutic agent, a diagnostic agent or a pharmaceutical agent. A therapeutic or pharmaceutical agent is one that alone or together with an additional compound induces the desired response (such as inducing a therapeutic or prophylactic effect when administered to a subject, including inhibiting or treating an ischemic event, such as a stroke). In some examples, the therapeutic agent includes an isolated microRNA (miRNA) that is down-regulated in patients with an a brain hemorrhage, such as a subarachnoid hemorrhage (SAH) or intracerebral hemorrhage (ICH).

Alteration in expression: An alteration in expression of a miRNA gene product refers to a change or difference, such as an increase or decrease, in the level of the miRNA gene product, that is detectable in a biological sample (such as a sample from subject, for example a serum sample), for example relative to a control, such as a subject not suffering a brain hemorrhage. An “alteration” in expression includes an increase in expression (up-regulation) or a decrease in expression (down-regulation). In some examples, an alteration in expression includes a change or difference, such as an increase or decrease, in the conversion of the information encoded in a miRNA gene into miRNA gene product. In some examples, the difference is relative to a control or reference value, such as an amount of miRNA expression in a sample from a healthy control subject.

Antisense compound: An oligomeric compound that is at least partially complementary to the region of a target nucleic acid molecule (such as a miRNA) to which it hybridizes. As used herein, an antisense compound that is “specific for” a target nucleic acid molecule is one which specifically hybridizes with and modulates expression of the target nucleic acid molecule. As used herein, a “target” nucleic acid is a nucleic acid molecule to which an antisense compound is designed to specifically hybridize and modulate expression. In some examples, the target nucleic acid molecule is a miRNA gene product.

Nonlimiting examples of antisense compounds include primers, probes, antisense oligonucleotides, siRNAs, miRNAs, shRNAs and ribozymes. As such, these compounds can be introduced as single-stranded, double-stranded, circular, branched or hairpin compounds and can contain structural elements such as internal or terminal bulges or loops. Double-stranded antisense compounds can be two strands hybridized to form double-stranded compounds or a single strand with sufficient self-complementarity to allow for hybridization and formation of a fully or partially double-stranded compound. In particular examples herein, the antisense compound is an antisense oligonucleotide, siRNA or ribozyme.

In some examples, an antisense compound is an “antisense oligonucleotide.” An antisense oligonucleotide is a single-stranded antisense compound that is a nucleic acid-based oligomer. An antisense oligonucleotide can include one or more chemical modifications to the sugar, base, and/or internucleoside linkages. Generally, antisense oligonucleotides are “DNA-like” such that when the antisense oligonucleotide hybridizes to a target RNA molecule, the duplex is recognized by RNase H (an enzyme that recognizes DNA:RNA duplexes), resulting in cleavage of the RNA.

In some embodiments an antisense compound is an miRNA mimic, for example to replenish the loss of such miRNA to reestablish control of gene regulation. Inhibitors of miRNAs are also contemplated.

Array: An arrangement of molecules, such as biological macromolecules (such nucleic acid molecules, for example probes), in addressable locations on or in a substrate. A “microarray” is an array that is miniaturized so as to require or be aided by microscopic examination for evaluation or analysis. Arrays are sometimes called DNA chips or biochips.

The array of molecules (“features”) makes it possible to carry out a very large number of analyses on a sample at one time. In certain example arrays, one or more molecules (such as an oligonucleotide probe) will occur on the array a plurality of times (such as twice), for instance to provide internal controls. The number of addressable locations on the array can vary, for example from at least 2, at least 5, at least 10, at least 14, at least 15, at least 20, at least 30, at least 50, at least 75, at least 100, at least 150, at least 200, at least 300, at least 500, least 550, at least 600, at least 800, at least 1000, at least 10,000, or more. In a particular example, an array includes 5-1000 addressable locations, such as 10-100 addressable locations. In particular examples, an array consists essentially of probes or primers (such as those that permit amplification) specific for the miRNA gene products discussed herein.

Within an array, each arrayed sample is addressable, in that its location can be reliably and consistently determined within at least two dimensions of the array. The feature application location on an array can assume different shapes. For example, the array can be regular (such as arranged in uniform rows and columns) or irregular. Thus, in ordered arrays the location of each sample is assigned to the sample at the time when it is applied to the array, and a key may be provided in order to correlate each location with the appropriate target or feature position. Often, ordered arrays are arranged in a symmetrical grid pattern, but samples could be arranged in other patterns (such as in radially distributed lines, spiral lines, or ordered clusters). Addressable arrays usually are computer readable, in that a computer can be programmed to correlate a particular address on the array with information about the sample at that position (such as hybridization or binding data, including for instance signal intensity). In some examples of computer readable formats, the individual features in the array are arranged regularly, for instance in a Cartesian grid pattern, which can be correlated to address information by a computer.

Biological sample: A biological specimen containing genomic DNA, RNA (including mRNA and microRNA), protein, or combinations thereof, obtained from a subject. Examples include, but are not limited to, saliva, peripheral blood, urine, tissue biopsy, surgical specimen, and autopsy material. In embodiments, the biological sample is blood, or a component thereof, such as plasma or serum. In embodiments, the biological sample is cerebrospinal fluid (CSF).

cDNA (complementary DNA): A piece of DNA lacking internal, non-coding segments (intrans) and regulatory sequences which determine transcription. cDNA can be synthesized by reverse transcription from RNA extracted from cells.

Contacting: Placement in direct physical association, including both a solid and liquid form. Contacting an agent with a cell can occur in vitro by adding the agent to isolated cells or in vivo by administering the agent to a subject.

Control: A “control” refers to a sample or standard used for comparison with a test sample, such as a sample obtained from a subject or patient (or plurality of patients). In some embodiments, the control is a sample obtained from a healthy patient (or plurality of patients) (also referred to herein as a “normal” control). In some embodiments, the control is a historical control or standard value (e.g. a previously tested control sample or group of samples that represent baseline or normal values, such as baseline or normal values in a normal subject or subject in which does not have a brain hemorrhage). In some examples the control is a standard value representing the average value (or average range of values) obtained from a plurality of patient samples (such as an average value or range of values of miRNA expression from normal patients).

Decrease or downregulate: To reduce the quality, amount, or strength of something. In some examples, when used in reference to the expression of nucleic acid molecules (such as a miRNA), a reduction or downregulation refers to any process which results in a decrease in production of a gene product. Gene downregulation includes any detectable decrease in the production of a gene product. In certain examples, production of a miRNA, or a decreases by at least 2-fold, at least 3-fold, at least 4-fold, at least 5-fold, at least 6-fold, at least 8-fold, at least 10-fold, at least 15-fold, at least 20-fold, at least 30-fold or at least 40-fold, as compared to a control.

Diagnosis: The process of identifying a disease by its signs, symptoms and/or results of various tests. The conclusion reached through that process is also called “a diagnosis.” Forms of testing commonly performed include blood tests, medical imaging, genetic analysis, urinalysis, biopsy and the methods disclosed herein.

Diagnostically significant amount: As used herein a “diagnostically significant amount” refers to an increase or decrease in the level of a miRNA gene product or ratio thereof in a biological sample that is sufficient to allow one to distinguish one patient population from another (such as a subject suffering an brain hemorrhage from one that is not). In some embodiments, the diagnostically significant increase or decrease is at least 2-fold, at least 3-fold, at least 4-fold, at least 5-fold, at least 6-fold, at least 8-fold, at least 10-fold, at least 15-fold, at least 20-fold, at least 30-fold or at least 40-fold relative to a control. In some embodiments, the diagnostically significant increase or decrease is at least 2-fold, at least 3-fold, at least 4-fold, at least 5-fold, at least 6-fold, at least 8-fold, at least 10-fold, at least 15-fold, at least 20-fold, at least 30-fold or at least 40-fold change in the ratio of two or more biomarkers relative to a control.

Effective amount: An amount of agent that is sufficient to generate a desired response, such as reducing or inhibiting one or more signs or symptoms associated with a condition or disease. When administered to a subject, a dosage will generally be used that will achieve target tissue concentrations. In some examples, an “effective amount” is one that treats one or more symptoms and/or underlying causes of any of a disorder or disease.

Measuring the level of expression: As used herein, measuring the level of expression of a particular miRNA refers to quantifying the amount of the miRNA present in a sample. Quantification can be either numerical or relative. Detecting expression of the miRNA can be achieved using any method known in the art or described herein, such as by RT-PCR. Detecting expression of a miRNA includes detecting expression of either a mature form of the miRNA or a precursor form (i.e., a pri-miRNA or pre-miR) that is correlated with expression of the miRNA. Typically, miRNA detection methods involve sequence specific detection, such as by RT-PCR. miRNA-specific primers and probes can be designed using the precursor and mature miRNA nucleic acid sequences that are known in the art and disclosed herein.

In embodiments, the change detected is an increase or decrease in expression as compared to a control, such as a reference value or a healthy control subject. In some examples, the detected increase or decrease is an increase or decrease of at least two-fold compared with the control or standard. Controls or standards for comparison to a sample, for the determination of differential expression, include samples believed to be normal as well as laboratory values (e.g., range of values), even though possibly arbitrarily set, keeping in mind that such values can vary from laboratory to laboratory.

Label: An agent capable of detection, for example by ELISA, spectrophotometry, flow cytometry, or microscopy. For example, a label can be attached to a nucleic acid molecule or protein (indirectly or directly), thereby permitting detection of the nucleic acid molecule or protein. Examples of labels include, but are not limited to, radioactive isotopes, enzyme substrates, co-factors, ligands, chemiluminescent agents, fluorophores, haptens, enzymes, and combinations thereof. Methods for labeling and guidance in the choice of labels appropriate for various purposes are discussed for example in Sambrook et al. (Molecular Cloning: A Laboratory Manual, Cold Spring Harbor, New York, 1989) and Ausubel et al. (In Current Protocols in Molecular Biology, John Wiley & Sons, New York, 1998).

Primer: a short nucleic acid molecule, for instance DNA oligonucleotides 10-100 nucleotides in length, such as 5, 6, 7, 8, 9, 10, 11, 12, or more in length. Primers can be annealed to a complementary target nucleic acid strand by nucleic acid hybridization to form a hybrid between the primer and the target nucleic acid strand. Primers can be used for amplification of a nucleic acid sequence, such as by PCR or other nucleic acid amplification methods known in the art.

Probe: A short sequence of nucleotides, such as at least 8, at least 10, at least 15, at least 20, or at least 21 nucleotides in length, used to detect the presence of a complementary sequence by molecular hybridization. In particular examples, oligonucleotide probes include a label that permits detection of oligonucleotide probe: target sequence hybridization complexes. Laboratory standards and values can be set based on a known or determined population value and can be supplied in the format of a graph or table that permits comparison of measured, experimentally determined values.

MicroRNA (miRNA or miR): A single-stranded RNA molecule that regulates gene expression in plants, animals and viruses. A gene encoding a microRNA is transcribed to form a primary transcript microRNA (pri-miR), which is processed to form a short stem-loop molecule, termed a precursor microRNA (pre-miR), followed by endonucleolytic cleavage to form the mature microRNA. Mature microRNAs are approximately 21-23 nucleotides in length and are partially complementary to the 3′UTR of one or more target messenger RNAs (mRNAs). The term “microRNA gene product” includes pri-miRs, pre-miRs and mature microRNAs (including minor mature miRNA species referred to as miR*). MicroRNAs modulate gene expression by promoting cleavage of target mRNAs or by blocking translation of the cellular transcript.

Patient or Subject: A term that includes human and non-human animals, such as those having arterial plaques. In one example, the patient or subject is a mammal, such as a human. “Patient” and “subject” are used interchangeably herein.

Pharmaceutically acceptable vehicles: The pharmaceutically acceptable carriers (vehicles) useful in this disclosure are conventional. Remington's Pharmaceutical Sciences, by E. W. Martin, Mack Publishing Co., Easton, Pa., 19th Edition (1995), describes compositions and formulations suitable for pharmaceutical delivery of one or more therapeutic compounds, molecules or agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Small interfering RNA (siRNA): A double-stranded nucleic acid molecule that modulates gene expression through the RNAi pathway (see, for example, Bass, Nature 411:428-9, 2001; Elbashir et al., Nature 411:494-8, 2001; and PCT Publication Nos. WO 00/44895;WO 01/36646;WO 99/32619;WO 00/01846;WO 01/29058;WO 99/07409; and WO 00/44914). siRNA molecules are generally 20-25 nucleotides in length with 2-nucleotide overhangs on each 3′ end. However, siRNAs can also be blunt ended. Generally, one strand of a siRNA molecule is at least partially complementary to a target nucleic acid, such as a target miRNA. siRNAs are also referred to as “small inhibitory RNAs,” “small interfering RNAs” or “short inhibitory RNAs.” As used herein, siRNA molecules need not be limited to those molecules containing only RNA, but further encompasses chemically modified nucleotides and non-nucleotides having RNAi capacity or activity. In an example, a siRNA molecule is one that reduces or inhibits the biological activity or expression of a miRNA gene product.

Treating a disease: A phrase referring to a therapeutic intervention that ameliorates a sign or symptom of a disease or pathological condition after it has begun to develop.

Upregulated or activated: When used in reference to the expression of a nucleic acid molecule (such as a miRNA), refers to any process which results in an increase in production of a gene product. In the context of the present disclosure, a gene product can be a primary transcript ncRNA, precursor ncRNA, or a mature ncRNA, such as a miRNA. Gene upregulation or activation includes any detectable increase in any of these molecules. In certain examples, production of a ncRNA such as a miRNA increases by at least 2-fold, at least 3-fold, at least 4-fold, at least 5-fold, at least 6-fold, at least 8-fold, at least 10-fold, at least 15-fold, at least 20-fold, at least 30-fold or at least 40-fold, as compared to a control.

Stroke: A medical condition in which poor blood flow to the brain results in cell death. There are two main types of stroke: ischemic, due to lack of blood flow, and hemorrhagic, due to bleeding. The two types of hemorrhagic strokes are intracerebral (within the brain) hemorrhage or subarachnoid hemorrhage. Subarachnoid hemorrhage (SAH) is bleeding into the subarachnoid space-the area between the arachnoid membrane and the pia mater surrounding the brain. SAH may occur as a result of a head injury or spontaneously, usually from a ruptured cerebral aneurysm. Spontaneous SAH occurs in about one per 10,000 people per year and comprises about 5 percent of all strokes. Intracerebral hemorrhage (ICH): is bleeding within the brain tissue itself. ICH makes up roughly 5% of the strokes.

### Overview of Several Embodiments

Subarachnoid hemorrhage (SAH) and intracerebral hemorrhage (ICH) are associated with different clinical outcome and possibly different miRNA profiles in cerebrospinal fluid (CSF) and plasma (PL). MicroRNAs (miRNAs) are key post-transcriptional regulators of gene expression. miRNAs regulate gene expression and protein translation and can thus provide new venues for therapeutic targets. miRNAs are non-coding RNAs (ncRNAs), a novel class of endogenous small RNAs that negatively regulate gene expression via degradation or translational inhibition of their target transcripts (mRNAs).

Recent investigations evaluating the role of miRNAs in the pathophysiology of cerebral injury have suggested that miRNA profiles change in relation to cerebrovascular diseases, such as ischemic and hemorrhagic stroke. Some miRNAs appear differentially expressed in the CSF as compared to blood in ICH patients. The relationship between circulating miRNA in spontaneous ICH and SAH is not well understood. As disclosed herein the inventors have determined differences in miRNA expression profile in CSF and blood (plasma) of patients with acute spontaneous ICH and SAH.

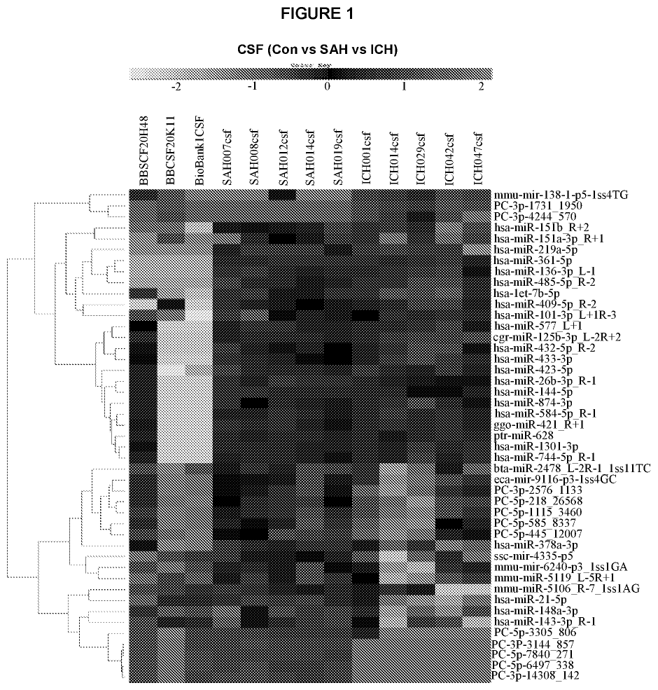

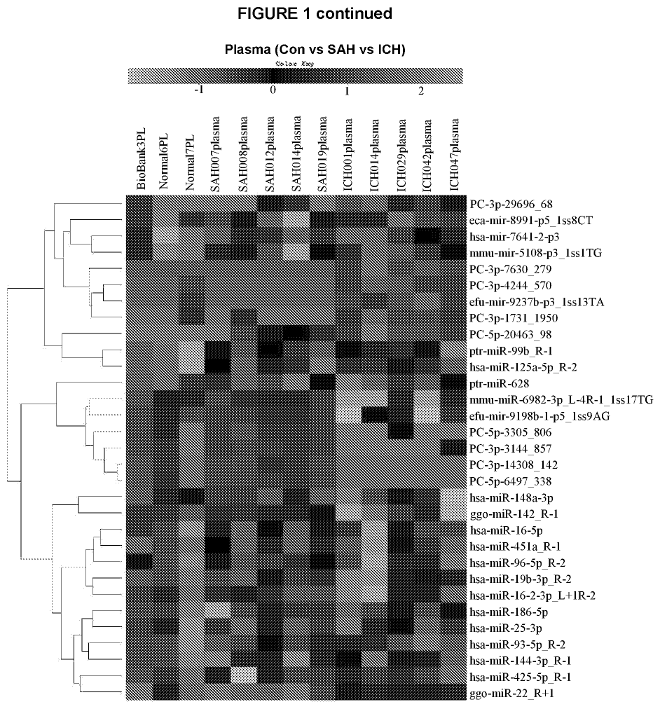

In CSF, 21 known and 4 novel miRNA highly expressed in the controls but reduced in SAH and ICH were identified (Table 1; FIG. 1). In plasma, 11 miRNA were downregulated in SAH and ICH compared to control. In CSF of SAH vs ICH, 5 miRNA were found at higher levels in SAH than ICH. In plasma, only 1 significant miRNA was identified. Pathway enrichment analysis found axon guidance for upregulated miRNA and cell adhesion for downregulated miRNA in SAH and ICH CSF. In plasma, enrichment for Ras signaling, axon guidance, platelet activation, and hemophilic cell adhesion were found.

Clinically, measuring these miRNAs in subjects presenting with stroke symptoms or risk factors associated with stroke, such as hypertension, has the advantage of determining whether, the patients has a brain hemorrhage, such as SAH and ICH, and/or distinguishing between SAH and ICH. The ability to makes these determinations may greatly improve patient outcomes and facilitate the correct treatment. For example, the ability to definitely distinguish between a hemorrhagic stroke and a ischemic stroke could determine whether recombinant tissue plasminogen activator (tPA) is given or surgical intervention to repair a compromised blood vessel is the preferred course of treatment. A rapid diagnostic method, such as disclosed herein, would have the potential to minimize diagnostic delays and improve outcomes. It also has tremendous cost-saving to the health system as it would identify the source of the stroke prior to an expensive clinical work-up, including computed tomography (CT) scans of the head, magnetic resonance imaging (MRI) of the neck and brain, CT angiography, etc. Consistent with the inventors discoveries, the inventors have developed novel methods for identifying and measuring biomarkers to predict and/or detect hemorrhagic stroke, such as SAH and ICH, and/or distinguishing between SAH and ICH. In addition, method of treatment, assay, kits and the like have been developed based on the inventors discoveries. Methods of Detecting Brain hemorrhage

As disclosed herein the differential level of expression of the miRNAs as disclosed in the Tables 1, 2, 3, and/or 4 below can be used to diagnose a brain hemorrhage such as one or more of ICH and SAH. Thus provided herein is a method that includes measuring at least one of miRNAs listed in Tables 1, 2, 3, and/or 4 in a cerebral spinal fluid sample and/or a plasma fluid sample obtained from a subject, wherein the at least one of the miRNAs comprise at least one of PC-5P-585_8337, PC-5P-218_26568, PC-5P-1115_3460 or PC-5P-445_12007. In embodiments, the method further includes selecting a subject with, or believed to have, suffered a stroke. In embodiments, the method is used for diagnosing or prognosis of a subject with stroke. In embodiments, the method is a method of diagnosing a subject with subarachnoid hemorrhage (SAH) and intracerebral hemorrhage (ICH).

In embodiments, the at least one miRNAs further comprises one or more of hsa-miR-21-5p, hsa-miR-423-5p, hsa-miR-451a_R-1, hsa-miR-338-5p R-1, hsa-miR-16-5p, hsa-miR-204-5p, hsa-miR-100-5p_R-1, hsa-miR-151a-5p, hsa-miR-144-3p R-1, hsa-miR-486-5p, hsa-miR-191-5p, hsa-miR-320a_R-2, hsa-miR-125a-5p R-2m hsa-miR-126-3p R-1, hsa-let-7b-5p, hsa-miR-186-5p, hsa-miR-125b-5p R-2, hsa-miR-26a-5p, and hsa-miR-92b-3p, and measuring comprises measuring an increase in the expression of hsa-miR-423-5p, hsa-miR-451a_R-1, hsa-miR-338-5p_R-1, hsa-miR-16-5p, hsa-miR-204-5p, hsa-miR-100-5p R-1, hsa-miR-151a-5p, hsa-miR-144-3p R-1, hsa-miR-486-5p, hsa-miR-191-5p, hsa-miR-320a_R-2, hsa-miR-125a-5p R-2, hsa-let-7b-5p, hsa-miR-186-5p, hsa-miR-125b-5p R-2, hsa-miR-26a-5p, hsa-miR-92b-3p, PC-5p-218_26568, hsa-miR-191-5p, in the cerebral spinal fluid sample and/or a decrease in hsa-miR-21-5p, PC-5p-585_8337, PC-5p-218_26568, PC-5p-1115_3460 or PC-5p-445_12007 hsa-miR-204-5p, hsa-miR-126-3p R-1 in the cerebral spinal fluid sample.

In embodiment the at least one miRNA further comprises one or more of hsa-miR-21-5p, hsa-miR-423-5p, hsa-miR-451a_R-1, hsa-miR-338-5p_R-1, hsa-miR-16-5p, hsa-miR-204-5p, hsa-miR-100-5p R-1, hsa-miR-151a-5p, hsa-miR-144-3p R-1, hsa-miR-486-5p, hsa-miR-191-5p, hsa-miR-320a_R-2, hsa-miR-125a-5p R-2m hsa-miR-126-3p R-1, hsa-let-7b-5p, hsa-miR-186-5p, hsa-miR-125b-5p R-2, hsa-miR-26a-5p, and hsa-miR-92b-3p, and measuring comprises measuring an increase in the expression of hsa-miR-423-5p, hsa-miR-338-5p R-1, hsa-miR-204-5p, hsa-miR-100-5p_R-1, hsa-miR-151a-5p, hsa-miR-486-5p, hsa-miR-191-5p, hsa-miR-125a-5p_R-2, hsa-miR-125b-5p R-2, hsa-miR-26a-5p, PC-5p-585_8337, hsa-miR-92b-3p, and PC-5p-445_12007, or hsa-let-7b-5p, hsa-miR-125b-5p R-2 in the plasma fluid sample and/or a decrease in hsa-miR-21-5p, hsa-miR-451a_R-1, hsa-miR-16-5p, hsa-miR-144-3p_R-1, hsa-miR-320a_R-2, hsa-miR-126-3p R-1, hsa-let-7b-5p, hsa-miR-186-5p, PC-5p-218_26568, and PC-5p-1115_3460, hsa-miR-486-5p, in the plasma fluid sample.

In embodiments, the method includes comparing the expression of one or more of hsa-miR-21-5p, hsa-miR-423-5p, hsa-miR-451a_R-1, hsa-miR-338-5p R-1, hsa-miR-16-5p, hsa-miR-204-5p, hsa-miR-100-5p R-1, hsa-miR-151a-5p, hsa-miR-144-3p R-1, hsa-miR-486-5p, hsa-miR-191-5p, hsa-miR-320a_R-2, hsa-miR-125a-5p R-2, hsa-miR-126-3p R-1, hsa-let-7b-5p, hsa-miR-186-5p, hsa-miR-125b-5p_R-2, hsa-miR-26a-5p, PC-5p-585_8337, hsa-miR-92b-3p, PC-5p-218_26568, PC-5p-1115_3460, and PC-5p-445_12007 to a control.

In embodiments, the expression of miR-hsa-miR-21-5p, hsa-miR-423-5p, hsa-miR-451a_R-1, hsa-miR-338-5p R-1, hsa-miR-16-5p, hsa-miR-204-5p, hsa-miR-100-5p R-1, hsa-miR-151a-5p, hsa-miR-144-3p R-1, hsa-miR-486-5p, hsa-miR-191-5p, hsa-miR-320a_R-2, hsa-miR-125a-5p R-2, hsa-miR-126-3p R-1, hsa-let-7b-5p, hsa-miR-186-5p, hsa-miR-125b-5p R-2, hsa-miR-26a-5p, PC-5p-585_8337, hsa-miR-92b-3p, PC-5p-218_26568, PC-5p-1115_3460, and PC-5p-445_12007 is detected with a probe and/or primers that respectively specifically bind to hsa-miR-21-5p, hsa-miR-423-5p, hsa-miR-451a_R-1, hsa-miR-338-5p_R-1, hsa-miR-16-5p, hsa-miR-204-5p, hsa-miR-100-5p R-1, hsa-miR-151a-5p, hsa-miR-144-3p R-1, hsa-miR-486-5p, hsa-miR-191-5p, hsa-miR-320a_R-2, hsa-miR-125a-5p_R-2, hsa-miR-126-3p_R-1, hsa-let-7b-5p, hsa-miR-186-5p, hsa-miR-125b-5p R-2, hsa-miR-26a-5p, PC-5p-585_8337, hsa-miR-92b-3p, PC-5p-218_26568, PC-5p-1115_3460, and PC-5p-445_12007, or an amplification product thereof. In embodiments, the probe and/or primers are labeled with a detectable label. In embodiments the expression of one or more of PC-3P-7630_279, PC-5P-1731_1950, PC-3P-4244_570, PC-3p-3144_857, PC-5p-6497_338. PC-5p-3305 806 is measured.

In embodiments, an increase in expression in cerebrospinal fluid (CSF) relative to a control of one or more of hsa-miR-423-5p, hsa-miR-451a_R-1, hsa-miR-338-5p R-1, hsa-miR-16-5p, hsa-miR-204-5p, hsa-miR-100-5p R-1, hsa-miR-151a-5p, hsa-miR-144-3p R-1, hsa-miR-486-5p, hsa-miR-191-5p, hsa-miR-320a_R-2,hsa-miR-125a-5p R-2, hsa-let-7b-5p, hsa-miR-186-5p, hsa-miR-125b-5p R-2, hsa-miR-26a-5p, hsa-miR-92b-3p, or PC-5p-218_26568, indicates that the subject has or is at risk of developing SAH. In embodiments, a decrease in expression in CSF relative to a control of one or more of hsa-miR-21-5p, hsa-miR-126-3p R-1, PC-5p-585_8337, PC-5p-218_26568, PC-5p-1115_3460 or PC-5p-445_12007, indicates that the subject has or is at risk of developing SAH. In embodiments, an increase in expression in CSF relative to a control of one or more of ICH hsa-miR-423-5p, hsa-miR-451a_R-1, hsa-miR-338-5p_R-1, hsa-miR-16-5p, hsa-miR-100-5p R-1, hsa-miR-151a-5p, hsa-miR-144-3p R-1, hsa-miR-486-5p, hsa-miR-191-5p, hsa-miR-320a_R-2, hsa-miR-125a-5p R-2, hsa-let-7b-5p, hsa-miR-186-5p, hsa-miR-125b-5p R-2, hsa-miR-26a-5p, hsa-miR-92b-3p, and PC-5p-218_26568, indicates that the subject has or is at risk of developing ICH. In embodiments, a decrease in expression in CSF relative to a control of one or more of hsa-miR-21-5p, hsa-miR-126-3p R-1, PC-5p-585_8337, PC-5p-218_26568, PC-5p-1115_3460 or PC-5p-445_12007, indicates that the subject has or is at risk of developing ICH.

In embodiments the method further includes distinguishing between SAH or ICH, wherein in CFS an increase in expression of hsa-miR-204-5p relative to a control indicates SAH and a decrease in expression of hsa-miR-204-5p relative to a control indicates ICH, and wherein in plasma an increase in expression of hsa-miR-486-5p relative to a control indicates SAH and a decrease in expression of hsa-miR-486-5p to a control indicates. ICH and a decrease in expression of hsa-let-7b-5p relative to a control indicates SAH and an increase in expression of hsa-let-7b-5p to a control indicates. ICH. In embodiments, an increase in expression in plasma relative to a control of one or more of hsa-miR-423-5p, hsa-miR-338-5p_R-1, hsa-miR-204-5p, hsa-miR-100-5p R-1, hsa-miR-151a-5p, hsa-miR-486-5p, hsa-miR-191-5p, hsa-miR-125a-5p R-2, hsa-miR-125b-5p R-2, hsa-miR-26a-5p, PC-5p-585_8337, hsa-miR-92b-3p, and PC-5p-445_12007, indicates that the subject has or is at risk of developing SAH. In embodiments, a decrease in expression in plasma relative to a control of one or more of hsa-miR-21-5p, hsa-miR-451a_R-1, hsa-miR-16-5p, hsa-miR-144-3p R-1, hsa-miR-320a_R-2, hsa-miR-126-3p R-1, hsa-let-7b-5p, hsa-miR-186-5p, PC-5p-218_26568, and PC-5p-1115_3460, indicates that the subject has or is at risk of developing SAH. In embodiments, an increase in expression in plasma relative to a control of one or more of ICH hsa-miR-423-5p, hsa-miR-338-5p_R-1, hsa-miR-204-5p, hsa-miR-100-5p R-1, hsa-miR-151a-5p hsa-miR-191-5p, hsa-miR-125a-5p_R-2, hsa-let-7b-5p, hsa-miR-125b-5p R-2, hsa-miR-26a-5p, PC-5p-585_8337, hsa-miR-92b-3p, and PC-5p-445_12007, indicates that the subject has or is at risk of developing ICH. In embodiments, a decrease in expression in CSF relative to a control of one or more of hsa-miR-21-5p, hsa-miR-451a_R-1, hsa-miR-16-5p, hsa-miR-144-3p R-1, hsa-miR-486-5p, hsa-miR-320a_R-2, hsa-miR-126-3p R-1, hsa-let-7b-5p, hsa-miR-186-5p, PC-5p-218_26568, and PC-5p-1115_3460, indicates that the subject has or is at risk of developing ICH. In embodiments, a miRNA detected in plasma and CSF of ICH patients and not detectable in plasma or CSF of SAH samples includes one or more of PC-3P-7630_279, PC-5P-1731_1950, or PC-3P-4244_570. The presence of these miRNA in plasma and/or CSF indicates ICH pathology, and not SAH pathology. In embodiments, a miRNA not detected in plasma or CSF of ICH patients, but detected in SAH plasma/CSF and control plasma/CSF samples includes one or more of PC-3p-3144_857, PC-5p-6497_338, or PC-5p-3305_806. Absence of these microRNA in plasma and CSF indicates ICH.

In addition to being able to detect or determine if a subject is at risk of developing a brain hemorrhage, such as ICH and SAH, the differential level of expression of the miRNAs as disclosed in the Tables 1 and 2 below can be used to discriminate between ICH and SAH. In embodiments, the method further includes distinguishing between SAH or ICH. In embodiments, in CFS the expression of hsa-miR-204-5p relative to a control can be used to discriminate SAH from ICH, for example, the expression of hsa-miR-204-5p is increased in SAH relative to a control and conversely a decreased in ICH relative to a control. In embodiments, in plasma the expression of hsa-miR-486-5p relative to a control can be used to discriminate SAH from ICH, for example, the expression of hsa-miR-486-5p is increased in SAH relative to a control and conversely a decreased in ICH relative to a control. In embodiments, in plasma the expression of hsa-miR-320a_R-2 and/or hsa-miR-320a_R-2 relative to a control can be used to discriminate SAH from ICH, for example, the expression of hsa-miR-320a_R-2 and/or hsa-miR-320a_R-2 is decreased in SAH relative to a control and conversely increased in ICH relative to a control.

In some embodiments of the methods, the diagnostically significant increase or decrease in expression of the miRNA gene product is at least a 1.1-fold, 1.2-fold, 1.3-fold, 1.4-fold, 1.5-fold, 2-fold, 3-fold, 4-fold, 5-fold, 10-fold increase or decrease, for example relative to the level of a control. In some example the ratio of one miRNA to another miRNA is at least 1.1, 1.2, 1.3, 1.4, 1.5, 2, 3, 4, 5, 6, 7, 8, 9, 10, 15, 20, 30, 50, 60, 70, 80, 100, or even greater. Thus in some embodiments, the ratio of the one miRNA to the another miRNA of at least 1.1, 1.2, 1.3, 1.4, 1.5, 2, 3, 4, 5, 6, 7, 8, 9, 10, 15, 20, 30, 50, 60, 70, 80, 100, or even greater is used as a control threshold, above which a brain hemorrhage, such as SAH and ICH, is diagnosed or detected.

In addition methods are disclosed herein may be used to find other biomarkers to predict and/or detect a brain hemorrhage, such as SAH and ICH. Comparing absolute or relative concentrations of miRNAs in the serum is complicated by differing blood volumes in subjects. To overcome this, normalization strategies may be used to determine the relative concentration (ratio) of one miRNA of interest to another. In this method, normalized cycle threshold (ΔCₜ) is calculated by one of the methods below. A direct ratio of two miRNAs of interest may be determined using the equation: ΔCₜ=C_{t,G1}−C_{t,G2}.

Where:
C_{t,G1}=Cycle threshold for ncRNA of interest 1; and
C_{t,G2}=Cycle threshold for ncRNA of interest 2.

Normalized ratios may be determined using the equation: ΔCₜ=(C_{t,G1}−C_{t,HK1})−(C_{t,G2}−C_{t,HK2}).

Where:
C_{t,G1}=Cycle threshold for miRNA of interest 1;
C_{t,G2}=Cycle threshold for miRNA of interest 2;
C_{t,HK1}=Cycle threshold for spike-in control RNA 1; and
C_{t,HK2}=Cycle threshold for spike-in control RNA 2.

The fold difference between the test ΔCₜ and a ΔCₜ for the control sample may be determined using the equation: Fold difference=2−(Δctₜₑₛₜ−Δct_{control})

Methods of detecting and measuring miRNA expression are known in the art and are described in detail below. In some examples, RT-PCR is used to measure the level of a miRNA, such as when a single miRNA is analyzed. In other cases, when multiple miRNA gene products are to be measured, it may be desirable to use microarray analysis.

The miRNA gene product measured can be a primary miRNA (pri-miR) precursor miRNA (pre-miR), or a mature miRNA (including minor mature miRNA products denoted miR*). In some examples, the nucleic acid sequences set forth as SEQ ID NOS; 1-4 above, or subsequences thereof or measured or determined.

In some embodiments of the methods, the biological sample is blood, or a component thereof, such as plasma or serum. In some examples, the biological sample is cerebrospinal fluid. Thus, the method in some examples includes obtaining an appropriate sample from the patient to be diagnosed or treated with the methods provided herein.

In some embodiments, the method further includes providing an appropriate therapy for the subject diagnosed with a brain hemorrhage, such as SAH and ICH. In some examples, the therapy includes administering an isolated miRNA gene product, such a miRNA gene product that has been identified as down-regulated in a brain hemorrhage, such as SAH and ICH, relative to a control. In some examples, the therapy includes administering an agent that inhibits expression of a miRNA gene product, such as an agent that inhibits a miRNA gene product identified as up-regulated in a brain hemorrhage, such as SAH and ICH, relative to a control. In other examples, the therapy includes administering an agent that reduces blood pressure to the subject. In some embodiments, the therapy includes surgical intervention or a recommendation of such intervention as known in the art.

In some embodiments, the method includes selecting a subject with, or believed to have, suffered a stroke. In some embodiments, the method is used for diagnosing or prognosing a subject with stroke. In some embodiments, the method includes selecting a subject with, or believed to have, suffered a brain hemorrhage, or at risk for such a stroke.

In some embodiments, once a patient's diagnosis is determined, an indication of that diagnosis can be displayed and/or conveyed to a clinician or other caregiver. For example, the results of the test are provided to a user (such as a clinician or other health care worker, laboratory personnel, or patient) in a perceivable output that provides information about the results of the test. In some examples, the output is a paper output (for example, a written or printed output), a display on a screen, a graphical output (for example, a graph, chart, voltammetric trace, or other diagram), or an audible output. In other examples, the output is a numerical value, such as an amount of miRNA expression in the sample or a relative amount of miRNA in the sample as compared to a control. In some examples, the numerical value is the ratio of expression of one or more miRNAs to one or more other miRNAs. In additional examples, the output is a graphical representation, for example, a graph of the expression and/or ratio of expression on a standard curve or ROG. In a particular example, the output (such as a graphical output) shows or provides a cut-off value or level that indicates the presence of a brain hemorrhage, such as SAH or ICH. In some examples, the output is communicated to the user, for example by providing an output via physical, audible, or electronic means (for example by mail, telephone, facsimile transmission, email, or communication to an electronic medical record). The output can provide quantitative information. In some examples, the output is accompanied by guidelines for interpreting the data, for example, numerical or other limits that indicate the presence or absence of a brain hemorrhage. The guidelines need not specify whether a brain hemorrhage is present or absent, although it may include such a diagnosis. The output, for example, can include normal or abnormal ranges or a cutoff, which the recipient of the output may then use to interpret the results, for example, to arrive at a diagnosis, prognosis, or treatment plan. In other examples, the output can provide a recommended therapeutic regimen. In some examples, the test may include determination of other clinical information.

In some embodiments, the disclosed methods of diagnosis include one or more of the following depending on the patient's diagnosis: a) prescribing a treatment regimen for the patient if the patient's determined diagnosis is considered to be positive for a brain hemorrhage, such as SAH or ICH; b) not prescribing a treatment regimen for the patient if the patient's determined diagnosis is considered to be negative for a brain hemorrhage, such as SAH or ICH; c) administering a treatment to the patient if the patient's determined diagnosis is considered to be positive for a brain hemorrhage, such as SAH or ICH; and d) not administering a treatment regimen to the patient if the patient's determined diagnosis is considered to be negative for a brain hemorrhage, such as SAH or ICH. In an alternative embodiment, the method can include recommending one or more of a)-d). Thus, disclosed is a method of treating a brain hemorrhage, such as SAH or ICH, in a subject.

### Kits and Assays

Also provided are kits and assays including at least two oligonucleotide probes and/or primers specific for a miRNA gene product, such as those described herein. In some embodiments, the probes and/or primers are labeled, with a detectable label. In some embodiments, the kits and assays include at least two oligonucleotide probes specific for hsa-miR-21-5p, hsa-miR-423-5p, hsa-miR-451a_R-1, hsa-miR-338-5p R-1, hsa-miR-16-5p, hsa-miR-204-5p, hsa-miR-100-5p R-1, hsa-miR-151a-5p, hsa-miR-144-3p R-1, hsa-miR-486-5p, hsa-miR-191-5p, hsa-miR-320a_R-2, hsa-miR-125a-5p R-2, hsa-miR-126-3p R-1, hsa-let-7b-5p, hsa-miR-186-5p, hsa-miR-125b-5p R-2, hsa-miR-26a-5p, PC-5p-585_8337, hsa-miR-92b-3p, PC-5p-218_26568, PC-5p-1115_3460, and PC-5p-445_12007. In some examples, the kits and assays include controls (such as positive and negative controls). In some examples, the probes and/or primers are present in an array. In some embodiments, the kits and assays include instructions for the use thereof. In embodiments, the probes are present in an array. In embodiments, an array includes a nucleic comprising a nucleic acid sequence that has the nucleic acid sequence set forth as one of SEQ ID NOS: 1, 2, 3, or 4 or complementary thereto.

### Detecting miRNA Expression

As described below, expression of one or more miRNAs associated with brain hemorrhage, such as SAH or ICH, can be detected using any one of a number of methods well known in the art. In some embodiments of the methods provided herein, miRNAs expression profiles are used to diagnose brain hemorrhage, such as SAH or ICH, and to predict the prognosis and develop potential therapies for patients with brain hemorrhage, such as SAH or ICH. Thus, the disclosed methods can include evaluating miRNAs, such as hsa-miR-21-5p, hsa-miR-423-5p, hsa-miR-451a_R-1, hsa-miR-338-5p R-1, hsa-miR-16-5p, hsa-miR-204-5p, hsa-miR-100-5p R-1, hsa-miR-151a-5p, hsa-miR-144-3p_R-1, hsa-miR-486-5p, hsa-miR-191-5p, hsa-miR-320a_R-2, hsa-miR-125a-5p R-2, hsa-miR-126-3p R-1, hsa-let-7b-5p, hsa-miR-186-5p, hsa-miR-125b-5p_R-2, hsa-miR-26a-5p, PC-5p-585_8337, hsa-miR-92b-3p, PC-5p-218_26568, PC-5p-1115_3460, and PC-5p-445_12007.

The sequences of precursor miRNAs are publicly available, such as through the miRBase database, available online by the University of Manchester, and formerly maintained by the Sanger Institute (see Griffiths-Jones et al., Nucleic Acids Res. 36:0154-0158, 2008; Griffiths-Jones et al., Nucleic Acids Res. 34:0140-0144, 2006; and Griffiths-Jones, Nucleic Acids Res. 32:0109-0111, 2004) and GENBANK®. In addition to the publically available miRNA sequence the sequence of the novel miRNAs as disclosed herein are provided as SEQ ID NOS: 1-4 above.

Any one of a number of methods for detecting expression of a gene of interest (including miRNA) known in the art can be used to detect expression of a miRNA. A number of these methods, including qRT-PCR, array, microarray, SAGE are described in further detail below. Detection and quantification of miRNA expression can be achieved by any one of a number of methods known in the art including those described herein. U.S. Patent Application Publication Nos. 2006/0211000 and 2007/0299030 describe methods of miRNA detection and quantification. Further, general methods for RNA extraction are well known in the art and are disclosed in standard textbooks of molecular biology, including Ausubel et al., Current Protocols of Molecular Biology, John Wiley and Sons (1997). Using the known sequences for a miRNA of interest, specific probes and primers can be designed for use in the detection methods described herein as appropriate.

In some cases, the miRNA detection method requires isolation of nucleic acid from a sample, such as a blood or CSF sample, for example a serum sample. Nucleic acids, including RNA and specifically miRNA, can be isolated using any suitable technique known in the art.

Microarray analysis of miRNA can be accomplished according to any method known in the art (see, for example, PCT Publication No. WO 2008/054828; Ye et al., Nat. Med. 9(4):416-423, 2003; Calin et al., N. Engl. J. Med. 353(17):1793-1801, 2005). In one example, RNA is extracted from a sample, the small RNAs (18-26-nucleotide RNAs) are size-selected from total RNA using denaturing polyacrylamide gel electrophoresis. Oligonucleotide linkers are attached to the 5′ and 3′ ends of the small RNAs and the resulting ligation products are used as templates for an RT-PCR reaction with 10 cycles of amplification. The sense strand PCR primer has a fluorophore attached to its 5′ end, thereby fluorescently labeling the sense strand of the PCR product. The PCR product is denatured and then hybridized to the microarray. A PCR product, referred to as the target nucleic acid that is complementary to the corresponding miRNA capture probe sequence on the array will hybridize, via base pairing, to the spot at which the capture probes are affixed. The spot will then fluoresce when excited using a microarray laser scanner. The fluorescence intensity of each spot is then evaluated in terms of the number of copies of a particular miRNA, using a number of positive and negative controls and array data normalization methods, which will result in assessment of the level of expression of a particular miRNA.

In an alternative method, total RNA containing miRNA extracted from a cell, biological fluid or tissue sample is used directly without size-selection of small RNAs, and 3′ end labeled using T4 RNA ligase and either a fluorescently-labeled short RNA linker. The RNA samples are labeled by incubation at 30° C. for 2 hours followed by heat inactivation of the T4 RNA ligase at 80° C. for 5 minutes. The fluorophore-labeled miRNAs complementary to the corresponding miRNA capture probe sequences on the array will hybridize, via base pairing, to the spot at which the capture probes are affixed. The microarray scanning and data processing is carried out.

Methods for quantitating RNA, including miRNA, are well known in the art. In some embodiments, the method utilizes RT-PCR. Generally, the first step in gene expression profiling by RT-PCR is the reverse transcription of the RNA template into cDNA, followed by its exponential amplification in a PCR reaction. Two commonly used reverse transcriptases are avian myeloblastosis virus reverse transcriptase (AMV-RT) and Moloney murine leukemia virus reverse transcriptase (MMLV-RT). However, any suitable reverse transcriptase known in the art can be used for RT-PCR. The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif.), following the manufacturer's instructions. The derived cDNA can then be used as a template in the subsequent PCR reaction.

Although the PCR step can use a variety of thermostable DNA-dependent DNA polymerases, it often employs the Taq DNA polymerase, which has a 5′-3′ nuclease activity but lacks a 3′-5′ proofreading endonuclease activity. TaqMan® PCR typically utilizes the 5′-nuclease activity of Taq or Tth DNA polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

To minimize errors and the effect of sample-to-sample variation, RT-PCR can be performed using an internal standard. The ideal internal standard is expressed at a constant level among different tissues, and is unaffected by the experimental treatment. RNAs commonly used to normalize patterns of gene expression are mRNAs for the housekeeping genes glyceraldehyde-3-phosphate-dehydrogenase (GAPDH), beta-actin, and 18S ribosomal RNA.

Primers that can be used to amplify a particular miRNA can be designed and synthesized according to well-known methods using publically available sequences of the miRNA as well as those provided herein.

SAGE is another method that allows the simultaneous and quantitative analysis of a large number of gene transcripts, without the need of providing an individual hybridization probe for each transcript. First, a short sequence tag (about 10-14 base pairs) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag (see, for example, Velculescu et al., Science 270:484-7, 1995; and Velculescu et al., Cell 88:243-51, 1997).

In particular embodiments provided herein, arrays can be used to evaluate miRNA expression, for example to detect a brain hemorrhage, such as SAH or ICH. When describing an array that comprises probes or primers specific for a particular set of miRNA, such an array includes probes or primers specific for the recited miRNAs (such as hsa-miR-21-5p, hsa-miR-423-5p, hsa-miR-451a_R-1, hsa-miR-338-5p R-1, hsa-miR-16-5p, hsa-miR-204-5p, hsa-miR-100-5p R-1, hsa-miR-151a-5p, hsa-miR-144-3p R-1, hsa-miR-486-5p, hsa-miR-191-5p, hsa-miR-320a_R-2, hsa-miR-125a-5p R-2, hsa-miR-126-3p R-1, hsa-let-7b-5p, hsa-miR-186-5p, hsa-miR-125b-5p R-2, hsa-miR-26a-5p, PC-5p-585_8337, hsa-miR-92b-3p, PC-5p-218_26568, PC-5p-1115_3460, and PC-5p-445_12007), and can further include control probes (for example to confirm the incubation conditions are sufficient). In one example, an array is a multi-well plate (e.g., 98 or 364 well plate). In some embodiments, the probe and/or primers are labeled with a detectable label, for example a fluorophore, isotope, enzyme, or any other moiety that is detectable.

### Modulating miRNA Expression for Treatment or Prevention of Brain Hemorrhage

It is disclosed herein that many miRNAs are differentially expressed in patients with a brain hemorrhagic, such as SAH or ICH or subject to such a brain hemorrhage, such as SAH or ICH. As such, an increase in the level of one or more miRNAs down-regulated in patients with a brain hemorrhage, such as SAH or ICH, or a decrease in the level of one or more miRNAs up-regulated in patients with a brain hemorrhage, such as SAH or ICH, or subject to such a brain hemorrhage, such as SAH or ICH may be beneficial for inhibiting the development or progression of the brain hemorrhage and/or for alleviating one or more signs or symptoms associated with the brain hemorrhage.

Without wishing to be bound by theory, it is believed that alterations in the level of one or more miRNA gene products can result in the deregulation of one or more intended targets for these miRNAs, which can lead to undesirable effects, such as inflammation response. Therefore, altering the level of the miRNA gene product (e.g., by increasing the level of a miRNA that is up-regulated or by decreasing the level of a miRNA that is down-regulated) may successfully treat or ameliorate one or more signs or symptoms of the disease, such as treat or ameliorate one or more signs or symptoms associated with the brain hemorrhage.

Provided herein is a method of treating a patient with a brain hemorrhage or subject to such a brain hemorrhage by administering to the patient a therapeutically effective amount of an agent that inhibits expression of a miRNA gene product that is up-regulated in patients with a brain hemorrhage, such as SAH or ICH or subject to such a brain hemorrhage compared with a control (such as a healthy control subject). Disclosed is a method of treating and/or preventing a brain hemorrhage, such as SAH or ICH or an associated inflammation response in a subject, including administering to the subject an effective amount of an agent that alters the expression of one or more of hsa-miR-21-5p, hsa-miR-423-5p, hsa-miR-451a_R-1, hsa-miR-338-5p R-1, hsa-miR-16-5p, hsa-miR-204-5p, hsa-miR-100-5p R-1, hsa-miR-151a-5p, hsa-miR-144-3p R-1, hsa-miR-486-5p, hsa-miR-191-5p, hsa-miR-320a_R-2, hsa-miR-125a-5p R-2, hsa-miR-126-3p R-1, hsa-let-7b-5p, hsa-miR-186-5p, hsa-miR-125b-5p R-2, hsa-miR-26a-5p, PC-5p-585_8337, hsa-miR-92b-3p, PC-5p-218_26568, PC-5p-1115_3460, and PC-5p-445_12007, thereby treating brain hemorrhage, such as SAH or ICH and/or administering an agent that increases the expression of at least one of hsa-miR-21-5p, hsa-miR-126-3p R-1, PC-5p-218_26568, and PC-5p-1115_3460. In some examples the agent that inhibits expression of a miRNA gene product is an antisense compound specific for hsa-miR-423-5p, hsa-miR-338-5p R-1, hsa-miR-100-5p R-1, hsa-miR-151a-5p, hsa-miR-191-5p, hsa-miR-125a-5p_R-2, hsa-miR-125b-5p R-2, hsa-miR-26a-5p, hsa-miR-92b-3p, such as an antisense oligonucleotide, siRNA or ribozyme.

In some embodiments, a method of treating and/or preventing subarachnoid hemorrhage (SAH) and intracerebral hemorrhage (ICH) or an associated inflammation response in a subject comprises administering to the subject an effective amount of an agent that alters the expression of one or more of hsa-miR-21-5p, hsa-miR-423-5p, hsa-miR-451a_R-1, hsa-miR-338-5p_R-1, hsa-miR-16-5p, hsa-miR-204-5p, hsa-miR-100-5p R-1, hsa-miR-151a-5p, hsa-miR-144-3p R-1, hsa-miR-486-5p, hsa-miR-191-5p, hsa-miR-320a_R-2, hsa-miR-125a-5p_R-2, hsa-miR-126-3p_R-1, hsa-let-7b-5p, hsa-miR-186-5p, hsa-miR-125b-5p R-2, hsa-miR-26a-5p, PC-5p-585_8337, hsa-miR-92b-3p, PC-5p-218_26568, PC-5p-1115_3460, and PC-5p-445_12007, thereby treating arterial plaque a brain hemorrhage. In embodiments, the agent decreases expression of hsa-miR-423-5p, hsa-miR-338-5p_R-1, hsa-miR-100-5p_R-1, hsa-miR-151a-5p, hsa-miR-191-5p, hsa-miR-125a-5p R-2, hsa-miR-125b-5p_R-2, hsa-miR-26a-5p, hsa-miR-92b-3p. In embodiments, the agent is an antisense compound specific for one of hsa-miR-423-5p, hsa-miR-338-5p R-1, hsa-miR-100-5p R-1, hsa-miR-151a-5p, hsa-miR-191-5p, hsa-miR-125a-5p R-2, hsa-miR-125b-5p_R-2, hsa-miR-26a-5p, hsa-miR-92b-3p. In embodiments, the antisense compound is an antisense oligonucleotide, siRNA or ribozyme. In embodiments, the method includes administering an agent that increases the expression of at least one of hsa-miR-21-5p, hsa-miR-126-3p R-1, PC-5p-218_26568, and PC-5p-1115_3460. In embodiments, the agent comprises one or more of hsa-miR-21-5p, hsa-miR-126-3p R-1, PC-5p-218_26568, and PC-5p-1115_3460, such as the nucleic acid set forth as one of SEQ ID NOS: 1, 2, 3, or 4.

As used herein, “inhibiting expression of a miRNA gene product” means that the production of the precursor and/or active, mature form of the miRNA gene product after treatment is less than the amount produced prior to treatment. Expression can be altered by decreasing the levels made or degrading the amount present to reduce the level. One skilled in the art can readily determine whether miRNA expression has been inhibited in a subject, using the techniques known in the art and described herein. Inhibition can occur at the level of gene expression (i.e., by inhibiting transcription of a miRNA gene encoding the miRNA gene product) or at the level of processing (e.g., by inhibiting processing of a miRNA precursor into a mature miRNA).

As used herein, a therapeutically effective amount of a compound that inhibits miRNA expression is an amount sufficient to result in a biological effect (such as alleviating one or more signs or symptoms of a brain. For example, an agent can decrease or increase the expression level of a target miRNA by a desired amount, for example by at least 2-fold, at least 3-fold, at least 4-fold, at least 5-fold, at least 6-fold, at least 8-fold, at least 10-fold, at least 15-fold, at least 20-fold, at least 30-fold or at least 40-fold relative to a control or reference value.

One skilled in the art can readily determine a therapeutically effective amount of an agent to be administered to a given subject by taking into account several factors, such as the size and weight of the subject; the extent of disease progression; the age, health and sex of the subject; the route of administration; and whether the administration is regional or systemic. One skilled in the art can also readily determine an appropriate dosage regimen for administering to a subject an agent that inhibits expression of miRNA gene product.

In some embodiments, a single agent that inhibits expression of a miRNA gene product is administered to the subject in need of treatment. In other embodiments, two or more agents (such as 2, 3, 4, 5, 6, 7, 8, 9 or 10 or more) that inhibit expression of a miRNA gene product are administered to the subject. When two or more agents are administered to the subject, the agents can be administered simultaneously (or within quick succession, such as within minutes of each other), or they can be administered at different times. For example, two or more agents can be administered one hour, twelve hours, one day, two days, five days, one week, two weeks or one month apart.

An agent that inhibits expression of a miRNA gene product can be any type of compound, such as, but not limited to, a nucleic acid molecule, polypeptide, antibody or small molecule, that is capable of inhibiting expression of one or more miRNA gene products. In some embodiments, the agent is an antisense compound.

Any type of antisense compound that specifically targets a miRNA gene product is contemplated for use to inhibit expression of the target miRNA gene product. In some examples, the agent is an antisense compound selected from an antisense oligonucleotide, a sRNA, or a ribozyme. Methods of designing, preparing and using antisense compounds are within the abilities of one of skill in the art. Furthermore, sequences for the disclosed miRNA gene products are publicly available. Antisense compounds specifically targeting a miRNA that is differentially expressed, (or other target nucleic acid) can be prepared by designing compounds that are complementary to the target nucleotide sequence, such as a pri-miRNA, pre-miRNA or mature miRNA sequence. Antisense compounds need not be 100% complementary to the target nucleic acid molecule to specifically hybridize with the target nucleic acid molecule. For example, the antisense compound, or antisense strand of the compound if a double-stranded compound, can be at least 75%, at least 80%, at least 85%, at least 90%, at least 95%, at least 99% or 100% complementary to the selected target nucleic acid sequence. Methods of screening antisense compounds for specificity are well known in the art (see, for example, U.S. Patent Application Publication No. 2003-0228689).

Generally, the principle behind antisense technology is that an antisense compound hybridizes to a target nucleic acid and effects the modulation of gene expression activity or function. The modulation of gene expression can be achieved by, for example, target RNA degradation or occupancy-based inhibition. An example of modulation of target RNA function by degradation is RNase H-based degradation of the target RNA upon hybridization with a DNA-like antisense compound, such as an antisense oligonucleotide.

Another example of modulation of gene expression by target degradation is RNA interference (RNAi) using small interfering RNAs (siRNAs). RNAi is a form of antisense-mediated gene silencing involving the introduction of double stranded (ds)RNA-like oligonucleotides leading to the sequence-specific reduction of targeted endogenous mRNA levels. Other compounds that are often classified as antisense compounds are ribozymes. Ribozymes are catalytic RNA molecules that can bind to specific sites on other RNA molecules and catalyze the hydrolysis of phosphodiester bonds in the RNA molecules. Ribozymes modulate gene expression by direct cleavage of a target nucleic acid, such as a miRNA gene product.

Each of the above-described antisense compounds provides sequence-specific target gene regulation. This sequence-specificity makes antisense compounds effective tools for the selective modulation of a target nucleic acid of interest, such as a miRNA gene product.

In some embodiments, the antisense compounds are antisense oligonucleotides. The miRNA gene product-specific antisense oligonucleotides can be any suitable length to allow for hybridization and modulation of gene expression. The length of an antisense oligonucleotide can vary, but is typically about 15 to about 40 nucleotides, including 15, 16, 17, 18, 19, 20, 21, 22, 23, 24, 25, 26, 27, 28, 29, 30, 31, 32, 33, 34, 35, 36, 37, 38, 39 or 40 nucleotides. In some embodiments, the antisense oligonucleotides are about 20 to about 35 nucleotides in length. The antisense oligonucleotides can be DNA, RNA or analogs thereof. Furthermore, the oligonucleotides provided herein can be unmodified or can comprise one or more modifications, such as modified internucleoside linkages, modified sugar moieties, modified bases, or a combination thereof. Oligonucleotide modifications are described in detail below.

In other embodiments, the antisense compounds are siRNA molecules. siRNAs useful for the disclosed methods include short double-stranded RNA from about 17 nucleotides to about 29 nucleotides in length, preferably from about 19 to about 25 nucleotides in length, such as about 21 to about 23 nucleotides in length. The siRNAs are made up of a sense RNA strand and a complementary antisense RNA strand annealed together by standard Watson-Crick base-pairing interactions. The sense strand includes a nucleic acid sequence that is substantially identical to a nucleic acid sequence contained within the target miRNA gene product. As used herein, an siRNA nucleic acid sequence that is “substantially identical” to a target sequence is a nucleic acid sequence that is identical to the target sequence, or that differs from the target sequence by one, two or three nucleotides. The sense and antisense strands of the siRNA can either include two complementary, single-stranded RNA molecules, or can be a single molecule having two complementary portions (which are base-paired) separated a single-stranded “hairpin” region.

The siRNA can also be altered RNA that differs from naturally occurring RNA by the addition, deletion, substitution and/or alteration of one or more nucleotides. Such alterations can include addition of non-nucleotide material, such as to one or both of the ends of the siRNA or to one or more internal nucleotides of the siRNA; modifications that make the siRNA resistant to nuclease digestion; or the substitution of one or more nucleotides in the siRNA with deoxyribonucleotides. One or both strands of the siRNA can also include a 3′ overhang. As used herein, a “3′ overhang” refers to at least one unpaired nucleotide extending from the 3′-end of a duplexed RNA strand. Thus, in certain embodiments, the siRNA includes at least one 3′ overhang of from 1 to about 6 nucleotides (which includes ribonucleotides or deoxyribonucleotides) in length, from 1 to about 5 nucleotides in length, from 1 to about 4 nucleotides in length, or from about 2 to about 4 nucleotides in length. In a particular embodiment, the 3′ overhang is present on both strands of the siRNA and is 2 nucleotides in length. For example, each strand of the siRNA can comprise 3′ overhangs of dithymidylic acid (“TT”) or diuridylic acid (“uu”).

In other embodiments, the antisense compound is a ribozyme. Ribozymes are nucleic acid molecules having a substrate binding region that is complementary to a contiguous nucleic acid sequence of a miRNA gene product, and which is able to specifically cleave the miRNA gene product. The substrate- binding region need not be 100% complementary to the target miRNA gene product. For example, the substrate-binding region can be, for example, at least about 50%, at least about 75%, at least about 85%, or at least about 95% complementary to a contiguous nucleic acid sequence in a miRNA gene product. The enzymatic nucleic acids can also include modifications at the base, sugar, and/or phosphate groups.

Antisense compounds, such as antisense oligonucleotides, siRNAs and ribozymes, can be produced chemically or biologically, or can be expressed from a recombinant plasmid or viral vector, as described in further detail below in regard to expression of isolated miRNA gene products. Exemplary methods for producing and testing antisense compounds are well known in the art (see, for example, U.S. Pat. Nos. 5,849,902 and 4,987,071; U.S. Patent Application Publication Nos. 2002/0173478 and 2004/0018176; Stein and Cheng, Science 261:1004, 1993; Werner and Uhlenbeck, Nucl. Acids Res. 23:2092-2096, 1995; Hammann et al., Antisense and Nucleic Acid Drug Dev. 9:25-31).

In some examples, the antisense compounds specific for a miRNA gene product contain one or more modifications to enhance nuclease resistance and/or increase activity of the compound. Modified antisense compounds include those comprising modified backbones or non-natural internucleoside linkages. As defined herein, oligonucleotides having modified backbones include those that retain a phosphorus atom in the backbone and those that do not have a phosphorus atom in the backbone.

Examples of modified oligonucleotide backbones include, but are not limited to, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkyl-phosphonates, thionoalkylphosphotriesters, and boranophosphates having normal 3′-5′ linkages, 2′-5′ linked analogs of these, and those having inverted polarity wherein the adjacent pairs of the nucleoside units are linked 3′-5′ to 5′-3′ or 2′-5′ to 5′-2′. Representative U.S. patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Examples of modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sultanate and sulfonamide backbones; amide backbones; and others having mixed N, O, S and CH2 component parts. Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

In some embodiments, both the sugar and the internucleoside linkage of the nucleotide units of the oligonucleotide or antisense compound are replaced with novel groups. One such modified compound is an oligonucleotide mimetic referred to as a peptide nucleic acid (PNA). In PNA compounds, the sugar-backbone of an oligonucleotide is replaced with an amide containing backbone, in particular an aminoethylglycine backbone. The bases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262. Further teaching of PNA compounds can be found in Nielsen et al. (Science 254, 1497-1500, 1991).

Modified oligonucleotides can also contain one or more substituted sugar moieties. In some examples, the oligonucleotides can comprise one of the following at the 2′ position: OH; F; O-, S-, or N-alkyl; O-, S-, or N-alkenyl; O-, S- or N- alkynyl; or O-alkyl-O-alkyl, wherein the alkyl, alkenyl and alkynyl may be substituted or unsubstituted C1 to C10 alkyl or C2 to C10 alkenyl and alkynyl. In other embodiments, the antisense compounds comprise one of the following at the 2′ position: C1 to C10 lower alkyl, substituted lower alkyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. In one example, the modification includes 2′-methoxyethoxy (also known as 2′-O-(2-methoxyethyl) or 2′-MOE) (Martin et al., Helv. Chim. Acta., 78, 486-504, 1995). In other examples, the modification includes 2′- dimethylaminooxyethoxy (also known as 2′-DMAOE) or 2′- dimethylaminoethoxyethoxy (also known in the art as 2′-O-dimethylaminoethoxyethyl or 2′-DMAEOE).

Similar modifications can also be made at other positions of the compound. Antisense compounds can also have sugar mimetics such as cyclobutyl moieties in place of the pentofuranosyl sugar. Representative United States patents that teach the preparation of modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920.

Oligonucleotides can also include base modifications or substitutions. As used herein, “unmodified” or “natural” bases include the purine bases adenine (A) and guanine (G), and the pyrimidine bases thymine (T), cytosine (C) and uracil (U). Modified bases include other synthetic and natural bases, such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further modified bases have been described (see, for example, U.S. Pat. No. 3,687,808; and Sanghvi, Y. S., Chapter 15, Antisense Research and Applications, pages 289-302, Crooke, S. T. and Lebleu, B., ed., CRC Press, 1993).

Certain of these modified bases are useful for increasing the binding affinity of antisense compounds. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2^{Q}C. Representative U.S. patents that teach the preparation of modified bases include, but are not limited to, U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,750,692.

Also provided is a method of treating a patient with a brain hemorrhage or subject to a brain hemorrhage by administering to the patient a therapeutically effective amount of an isolated miRNA gene product that is down-regulated in a patient with a brain hemorrhage or subject to such a brain hemorrhage, relative to a control (such as a healthy subject). For example, a subject with a brain hemorrhage or prone to a brain hemorrhage, is treated by administering a therapeutically effective amount of an isolated hsa-miR-21-5p, hsa-miR-126-3p R-1, PC-5p-218_26568, and PC-5p-1115_3460 gene product. As described herein, the miRNA gene product can be a pri-miRNA, a pre-miRNA or a mature miRNA.

In embodiments, the disclosed methods comprise administering an effective amount of at least one isolated miRNA gene product, or an isolated variant or biologically-active fragment thereof. The isolated miRNA gene product that is administered to the subject can be identical to an endogenous wild-type miRNA gene product (such as a pri-miRNA, pre-miRNA or mature miRNA) that is down-regulated, or it can be a variant or biologically-active fragment thereof. As defined herein, a “variant” of a miRNA gene product refers to a miRNA that has less than 100% identity to a corresponding wild-type miRNA gene product and possesses one or more biological activities of the corresponding wild-type miRNA gene product. Examples of such biological activities include, but are not limited to, inhibition of expression of a target RNA molecule (e.g., inhibiting translation of a target RNA molecule, modulating the stability of a target RNA molecule, or inhibiting processing of a target RNA molecule) and inhibition of a cellular process associated with a brain hemorrhage or subject to a brain hemorrhage. These variants include species variants and variants that are the consequence of one or more mutations (e.g., a substitution, a deletion, an insertion) in a miRNA gene. In certain embodiments, the variant is at least about 70%, at least about 75%, at least about 80%, at least about 85%, at least about 90%, at least about 95%, at least about 98%, or at about 99% identical to a corresponding wild-type miRNA gene product.

Disclosed is a composition that includes an miRNA oligonucleotide having the nucleic acid sequence set forth as one of SEQ ID NOS: 1, 2, 3, 4, 5, 6, 7, 8, 9, or 10. In embodiments, the composition includes a pharmaceutically acceptable carrier.

As used herein, a “biologically-active fragment” of a miRNA gene product refers to an RNA fragment of a miRNA gene product that possesses one or more biological activities of a corresponding wild-type miRNA gene product. As described above, examples of such biological activities include, but are not limited to, inhibition of expression of a target RNA molecule and inhibition of a cellular process associated with a brain hemorrhage or subject to a brain hemorrhage. In certain embodiments, the biologically-active fragment is at least about 9, at least about 11, at least about 13, at least about 15, at least about 17 or at least about 19 nucleotides in length.

A therapeutically effective amount of an isolated gene product can be, for example, the amount necessary to alleviate one or more signs or symptoms of a with a brain hemorrhage or subject to such a brain hemorrhage and/or the amount required to delay progression. One of skill in the art can determine the amount of an isolated miRNA gene product required for therapeutic efficacy.

In some embodiments, a single isolated miRNA gene product is administered to the subject in need of treatment. In other embodiments, two or more miRNA gene products (such as 2, 3, 4, 5, 6, 7, 8, 9 or 10 or more) are administered to the subject. When two or more miRNA gene products are administered to the subject, the miRNA gene products can be administered simultaneously (or within quick succession, such as within minutes of each other), or they can be administered at different times. For example, two or more miRNA gene products can be administered one hour, twelve hours, one day, two days, five days, one week, two weeks or one month apart.

In some embodiments, an isolated miRNA gene product can be administered to a subject in combination with one or more additional treatments for with a brain hemorrhage.

As used herein, an “isolated” miRNA gene product is one that is synthesized, or is purified away from other biological components of the cell or tissue in which the miRNA naturally occurs. For example, a synthetic miRNA gene product, or a miRNA gene product partially or completely separated from the other biological components of its natural state is considered to be “isolated.” Isolated miRNA gene products can be obtained using a number of standard techniques. For example, the miRNA gene products can be chemically synthesized or recombinantly produced using methods known in the art. In one embodiment, miRNA gene products are chemically synthesized using appropriately protected ribonucleoside phosphoramidites and a conventional DNA/RNA synthesizer. Commercial suppliers of synthetic RNA molecules or synthesis reagents include, for example, Proligo (Hamburg, Germany), Dharmacon Research (Lafayette, Colo.), Pierce Chemical (Rockford, Ill.), Glen Research (Sterling, VS), ChemGenes (Ashland, Mass.) and Cruachem (Glasgow, United Kingdom).

In some embodiments, the method includes administering a vector encoding a miRNA gene product. Vectors can be of non-viral (for example, plasmids) or viral (for example, adenovirus, adeno-associated virus, retrovirus, herpes virus, vaccinia virus) origin. Suitable vectors, such as gene therapy vectors, are well known in the art.

In some examples, the miRNA gene products are expressed from recombinant circular or linear DNA plasmids using any suitable promoter. Suitable promoters for expressing RNA from a plasmid include, for example, the U6 or H1 RNA pol III promoter sequences, or a cytomegalovirus promoter. Selection of other suitable promoters is within the skill in the art. The recombinant plasmids of the invention can also comprise inducible or regulatable promoters for expression of the miRNA gene products. In embodiments, a miRNA gene product includes a nucleic comprising a nucleic acid sequence that has the nucleic acid sequence set forth as one of SEQ ID NOS: 1, 2, 3, or 4 or complementary thereto.

When two or more miRNA gene products are to be expressed, the miRNA gene products can each be expressed from separate recombinant plasmids, or they can be expressed from the same recombinant plasmid. In one embodiment, the miRNA gene products are expressed as RNA precursor molecules from a single plasmid, and the precursor molecules are processed into the functional miRNA gene product within the target cell. Selection of plasmids suitable for expressing the miRNA gene products, methods for inserting nucleic acid sequences into the plasmid to express the gene products, and methods of delivering the recombinant plasmid to the cells of interest are within the skill in the art (see, for example, Zeng et al., Mol. Ce119:1327-1333, 2002; Tuschl, Nat. Biotechnol., 20:446-448, 2002; Brummelkarnp et al., Science 296:550-553, 2002; Miyagishi et al., Nat. Biotechnol. 20:497-500, 2002; Paddison et al., Genes Dev. 16:948-958, 2002; Lee et al., Nat. Biotechnol. 20:500-505, 2002; and Paul et al., Nat. Biotechnol. 20:505-508, 2002). In one embodiment, a plasmid expressing the miRNA gene product comprises a sequence encoding a miRNA precursor RNA operably linked to the CMV intermediate-early promoter.

The miRNA gene products can also be expressed from recombinant viral vectors. When administering two or more miRNA gene products, it is contemplated that the miRNA gene products can be expressed from two separate recombinant viral vectors, or from the same viral vector. The RNA expressed from the recombinant viral vectors can either be isolated from cultured cell expression systems by standard techniques, or can be expressed directly in target cells or tissues.

The recombinant viral vectors of use with the disclosed methods include sequences encoding the miRNA gene products and any suitable promoter for expressing the RNA sequences. Suitable promoters include, but are not limited to, the U6 or H1 RNA pol III promoter sequences, or a cytomegalovirus promoter. Selection of other suitable promoters is within the skill in the art. The recombinant viral vectors of the invention can also comprise inducible or regulatable promoters for expression of the miRNA gene products.

Suitable viral vectors include, but are not limited to, adenovirus vectors, adeno-associated virus vectors, retroviral vectors, lentiviral vectors, herpesviral vectors, and the like. For example, adenovirus vectors can be first, second, third and/or fourth generation adenoviral vectors or gutless adenoviral vectors. Adenovirus vectors can be generated to very high titers of infectious particles; infect a great variety of cells; efficiently transfer genes to cells that are not dividing; and are seldom integrated in the host genome, which avoids the risk of cellular transformation by insertional mutagenesis (Zern and Kresinam, Hepatology 25(2), 484-491, 1997). Representative adenoviral vectors which can be used for the methods provided herein are described by Stratford-Perricaudet et al. (J. Clin. Invest. 90: 626-630,1992); Graham and Prevec (In Methods in Molecular Biology: Gene Transfer and Expression Protocols 7: 109-128, 1991); and Barr et al. (Gene Therapy, 2:151-155,1995).

Adena-associated virus (AAV) vectors also are suitable for administration of HCC-associated genes. Methods of generating AAV vectors, administration of AAV vectors and their use are well known in the art (see, for example, U.S. Pat. No. 6,951,753; U.S. Pre-Grant Publication Nos. 2007-036757, 2006-205079, 2005-163756, 2005-002908; and PCT Publication Nos. WO 2005/116224 and WO 2006/119458).

Retrovirus, including lentivirus, vectors can also be used with the methods described herein. Lentiviruses include, but are not limited to, human immunodeficiency virus (such as HIV-1 and HIV-2), feline immunodeficiency virus, equine infectious anemia virus and simian immunodeficiency virus. Other retroviruses include, but are not limited to, human T-lymphotropic virus, simian T-lymphotropic virus, murine leukemia virus, bovine leukemia virus and feline leukemia virus. Methods of generating retrovirus and lentivirus vectors and their uses have been well described in the art (see, for example, U.S. Pat. Nos. 7,211,247; 6,979,568; 7,198,784; 6,783,977; and 4,980,289).

Suitable herpesvirus vectors can be derived from any one of a number of different types of herpesviruses, including, but not limited to, herpes simplex virus-1 (HSV-1), HSV-2 and herpesvirus saimiri. Recombinant herpesvirus vectors, their construction and uses are well described in the art (see, for example, U.S. Pat. Nos. 6,951,753; 6,379,674; 6,613,892; 6,692,955; 6,344,445; 6,319,703; and 6,261,552; and U.S. Patent Application Publication No. 2003-0083289).

One skilled in the art can readily determine an effective amount of a miRNA gene product to be administered to a given subject, by taking into account factors, such as the size and weight of the subject; the extent of disease progression; the age, health and sex of the subject; the route of administration; and whether the administration is regional or systemic.

For example, an effective amount of an isolated miRNA gene product can be based on the approximate body weight of a subject to be treated. Such effective amounts can be administered by any suitable route, such as, for example, intravenous or intraarterial. In some examples, an effective amount of the isolated miRNA gene product that is administered to a subject can range from about 5 to about 3000 micrograms/kg of body weight, from about 700 to about 1000 micrograms/kg of body weight, or greater than about 1000 micrograms/kg of body weight.

One skilled in the art can also readily determine an appropriate dosage regimen for the administration of an isolated miRNA gene product to a given subject. For example, a miRNA gene product can be administered to the subject once (e.g., as a single injection or deposition). Alternatively, a miRNA gene product can be administered once or twice daily to a subject for a period of from about three to about twenty-eight days, more particularly from about seven to about ten days. In a particular dosage regimen, a miRNA gene product is administered once a day for seven days. Where a dosage regimen comprises multiple administrations, it is understood that the effective amount of the miRNA gene product administered to the subject can comprise the total amount of gene product administered over the entire dosage regimen.

Agents can be administered to a subject in need of treatment using any suitable means known in the art. Methods of administration include, but are not limited to, intraductal, intradermal, intramuscular, intraperitoneal, parenteral, intravenous, subcutaneous, vaginal, rectal, intranasal, inhalation, oral or by gene gun. Intranasal administration refers to delivery of the compositions into the nose and nasal passages through one or both of the nares and can comprise delivery by a spraying mechanism or droplet mechanism, or through aerosolization of the nucleic acid or virus. Administration of the compositions by inhalant can be through the nose or mouth via delivery by spraying or droplet mechanisms. Delivery can be directly to any area of the respiratory system via intubation. Parenteral administration is generally achieved by injection. Injectables can be prepared in conventional forms, either as liquid solutions or suspensions, solid forms suitable for solution of suspension in liquid prior to injection, or as emulsions. Injection solutions and suspensions can be prepared from sterile powders, granules, and tablets. Administration can be systemic or local.

Agents can be administered in any suitable manner, preferably with pharmaceutically acceptable carriers. Pharmaceutically acceptable carriers are determined in part by the particular composition being administered, as well as by the particular method used to administer the composition. Accordingly, there is a wide variety of suitable formulations of pharmaceutical compositions of the present disclosure.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Administration can be accomplished by single or multiple doses. The dose required will vary from subject to subject depending on the species, age, weight and general condition of the subject, the particular therapeutic agent being used and its mode of administration. An appropriate dose can be determined by one of ordinary skill in the art using only routine experimentation.

In some embodiments, the therapeutic agent is a nucleic acid molecule, such as a miRNA gene product, a vector encoding a miRNA gene product, an antisense compound or a vector encoding an antisense compound. A nucleic acid-based therapeutic agent can be administered to a subject by any suitable route. In some examples, the agents are administered using an enteral or parenteral administration route. Suitable enteral administration routes include, for example, oral, rectal, or intranasal delivery. Suitable parenteral administration routes include, for example, intravascular administration (such as intravenous bolus injection, intravenous infusion, intra-arterial bolus injection, intra-arterial infusion and catheter instillation into the vasculature); subcutaneous injection or deposition, including subcutaneous infusion (such as by osmotic pumps); direct application to the tissue of interest, for example by a catheter or other placement device (e.g., a suppository or an implant comprising a porous, non-porous, or gelatinous material); and inhalation. Particularly suitable administration routes are injection, infusion and direct injection into a target tissue.

In the context of the present disclosure, a miRNA gene product or an antisense compound can be administered to the subject either as naked RNA or DNA in combination with a delivery reagent, or can be encoded by a recombinant plasmid or viral vector. Recombinant plasmids and viral vectors including sequences that express the miRNA gene products or antisense compounds, and techniques for delivering such plasmids and vectors to target cells, are well known in the art.

In some embodiments, liposomes are used to deliver a miRNA gene product or antisense compound (or nucleic acids comprising sequences encoding them) to a subject. Liposomes can also increase the blood half-life of the gene products or nucleic acids. Suitable liposomes for use in the invention can be formed from standard vesicle-forming lipids, which generally include neutral or negatively charged phospholipids and a sterol, such as cholesterol. The selection of lipids is generally guided by consideration of several factors, such as the desired liposome size and half-life of the liposomes in the blood stream. A variety of methods are known in the art for preparing liposomes (see, for example, Szoka et al., Ann. Rev. Biophys. Bioeng. 9:467, 1980; and U.S. Pat. Nos. 4,235,871; 4,501,728; 4,837,028; and 5,019,369). In some embodiments, polymers can be used to deliver a miRNA gene product or antisense compound to a subject. Cationic lipids and polymers that can be used to deliver therapeutic RNA molecules have been described (see, for example, Zhang et al., J Control Release. 123(1):1-10, 2007; Vorhies et al., Methods Mol Biol. 480:11-29, 2009; and U.S. Patent Application Publication No. 2009/0306194). Polypeptide carriers can also be used to administer a miRNA gene product to a subject (see, for example, Rahbek et al., J. Gene Med. 10:81-93, 2008).

Appropriate doses of small molecule agents depend upon a number of factors known to those or ordinary skill in the art, e.g., a physician. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

Also disclosed is a method of determining the effectiveness of an agent for treating and/or preventing a brain hemorrhage or an associated inflammation response in a subject. The method including detecting expression of at least one of hsa-miR-21-5p, hsa-miR-423-5p, hsa-miR-451a_R-1, hsa-miR-338-5p_R-1, hsa-miR-16-5p, hsa-miR-204-5p, hsa-miR-100-5p R-1, hsa-miR-151a-5p, hsa-miR-144-3p R-1, hsa-miR-486-5p, hsa-miR-191-5p, hsa-miR-320a_R-2, hsa-miR-125a-5p R-2, hsa-miR-126-3p_R-1, hsa-let-7b-5p, hsa-miR-186-5p, hsa-miR-125b-5p R-2, hsa-miR-26a-5p, PC-5p-585_8337, hsa-miR-92b-3p, PC-5p-218_26568, PC-5p-1115_3460, and PC-5p-445_12007 in a sample obtained from the subject following treatment with the agent and comparing expression of the at least one of hsa-miR-21-5p, hsa-miR-423-5p, hsa-miR-451a_R-1, hsa-miR-338-5p_R-1, hsa-miR-16-5p, hsa-miR-204-5p, hsa-miR-100-5p_R-1, hsa-miR-151a-5p, hsa-miR-144-3p R-1, hsa-miR-486-5p, hsa-miR-191-5p, hsa-miR-320a_R-2, hsa-miR-125a-5p R-2, hsa-miR-126-3p R-1, hsa-let-7b-5p, hsa-miR-186-5p, hsa-miR-125b-5p R-2, hsa-miR-26a-5p, PC-5p-585_8337, hsa-miR-92b-3p, PC-5p-218_26568, PC-5p-1115_3460, and PC-5p-445_12007 in a sample obtained from the subject following treatment to a reference value, wherein an alteration in the expression of the at least one of hsa-miR-21-5p, hsa-miR-423-5p, hsa-miR-451a_R-1, hsa-miR-338-5p R-1, hsa-miR-16-5p, hsa-miR-204-5p, hsa-miR-100-5p R-1, hsa-miR-151a-5p, hsa-miR-144-3p R-1, hsa-miR-486-5p, hsa-miR-191-5p, hsa-miR-320a_R-2, hsa-miR-125a-5p R-2, hsa-miR-126-3p R-1, hsa-let-7b-5p, hsa-miR-186-5p, hsa-miR-125b-5p R-2, hsa-miR-26a-5p, PC-5p-585_8337, hsa-miR-92b-3p, PC-5p-218_26568, PC-5p-1115_3460, and PC-5p-445_12007 following treatment indicates that the agent is effective for the agent for treating and/or preventing a brain hemorrhage or an associated inflammation response in the subject. In some embodiments, the reference value represents an expression value of the at least one of hsa-miR-21-5p, hsa-miR-423-5p, hsa-miR-451a_R-1, hsa-miR-338-5p R-1, hsa-miR-16-5p, hsa-miR-204-5p, hsa-miR-100-5p R-1, hsa-miR-151a-5p, hsa-miR-144-3p R-1, hsa-miR-486-5p, hsa-miR-191-5p, hsa-miR-320a_R-2, hsa-miR-125a-5p R-2, hsa-miR-126-3p R-1, hsa- let-7b-5p, hsa-miR-186-5p, hsa-miR-125b-5p R-2, hsa-miR-26a-5p, PC-5p-585_8337, hsa-miR-92b-3p, PC-5p-218_26568, PC-5p-1115_3460, and PC-5p-445_12007 in a sample from the subject prior to treatment with the agent.

### EXAMPLE

This example describes the determination of the ability of specific microRNAs (miRNAs) to serve as diagnostic biomarkers of subarachnoid hemorrhage (SAH) and intracerebral hemorrhage (ICH) as well as the ability to distinguish between SAH and ICH.

### Methods

Whole blood and CSF were collected from ICH and SAH patients (n=3 each) with 48 hrs. Non-ICH/SAH plasma and CSF controls (Cont, n=3 each) were also processed. Total RNA containing small RNAs were isolated from a 200 μl sample and used for small library construction and sequencing and analyzed for differentially expression (p<0.05) using the Ballgown Rpackage. Gene set enrichment analysis of KEGG pathways and Gene Ontology (GO) were done in miRWalk (p<0.01).

### Results

In CSF, 21 known and 4 novel miRNA highly expressed in the controls but reduced in SAH and ICH were identified (Table 1; FIG. 1). In plasma, 11 miRNA were downregulated in SAH and ICH compared to control. In CSF of SAH vs ICH, 5 miRNA were found at higher levels in SAH than ICH. In plasma, only 1 significant miRNA was identified. Pathway enrichment analysis found axon guidance for upregulated miRNA and cell adhesion for downregulated miRNA in SAH and ICH CSF. In plasma, enrichment for Ras signaling, axon guidance, platelet activation, and hemophilic cell adhesion were found.

TABLE 1

| | CSF (Mean Reads) | | | PLASMA (Mean Reads) | | |
|---|---|---|---|---|---|---|
| miRNA ID | Cont | SAH | ICH | Cont | SAH | ICH |
| hsa-miR-21-5p | 1,183 | 881 | 211 | 411 | 267 | 263 |
| hsa-miR-423-5p | 503 | 2,821 | 3,080 | 1,988 | 2,646 | 2,263 |
| hsa-miR-451a_R-1 | 1,469 | 6,286 | 7,444 | 31,521 | 7,909 | 4,890 |
| hsa-miR-338-5p_R-1 | 164 | 601 | 1,118 | 11 | 13 | 390 |
| hsa-miR-16-5p | 210 | 609 | 548 | 3,291 | 592 | 420 |
| hsa-miR-204-5p | 1,267 | 1,602 | 890 | 1 | 3 | 170 |
| hsa-miR-100-5p_R-1 | 1,296 | 2,816 | 6,948 | 296 | 503 | 2,395 |
| hsa-miR-151a-5p | 241 | 431 | 987 | 490 | 1,215 | 960 |
| hsa-miR-144-3p_R-1 | 90 | 482 | 590 | 2,861 | 646 | 655 |
| hsa-miR-486-5p | 1,264 | 3,854 | 7,828 | 10,349 | 11,353 | 7,807 |
| hsa-miR-191-5p | 936 | 1,386 | 2,350 | 2,853 | 9,100 | 3,903 |
| hsa-miR-320a_R-2 | 104 | 176 | 531 | 158 | 144 | 244 |
| hsa-miR-125a-5p_R-2 | 669 | 1,464 | 3,036 | 452 | 1,306 | 1,454 |
| hsa-miR-126-3p_R-1 | 492 | 81 | 133 | 627 | 499 | 416 |
| hsa-let-7b-5p | 457 | 1,460 | 3,817 | 1,161 | 599 | 1,845 |
| hsa-miR-186-5p | 462 | 1,086 | 686 | 892 | 383 | 366 |
| hsa-miR-125b-5p_R-2 | 2,790 | 5,843 | 12,657 | 444 | 738 | 4,783 |
| hsa-miR-26a-5p | 1,022 | 1,275 | 2,351 | 2,759 | 6,635 | 4,002 |
| PC-5p-585_8337 | 6,867 | 133 | 125 | 1,233 | 1,336 | 1,534 |
| hsa-miR-92b-3p | 120 | 318 | 1,304 | 37 | 73 | 731 |
| PC-5p-218_26568 | 13,945 | 1,137 | 366 | 3,651 | 3,529 | 3,549 |

Reads were mapped to species-specific genome in miRbase. Reads un-mapped to selected miRNAs in miRbase were subjected to mRNA, Rfam, and repbase. Reads un-mapped were then mapped to species-specific genome are analyzed for hairpin formation, sequences that potentially form hairpins are potential novel miRNA. The inventors identified four novel miRNAs in CSF and Plasma of SAH and ICH vs normal control CSF and Plasma (Table 2). All four miRNAs were decreased in CSF of SAH and ICH, and 1 miRNA was found to be higher in the plasma of SAH and ICH than control plasma.

TABLE 2

| REPORTER | CON | SAH | ICH | CON | SAH | ICH | |
|---|---|---|---|---|---|---|---|
| NAME | CSF | CSF | SAH | PL | PL | PL | MIR_SEQUENCE |
| PC-5P- | 6867 | 133 | 125 | 1233 | 1336 | 1534 | GCACCACGTTCCCGTGG |
| 585_8337 | | | | | | | (SEQ ID NO: 1) |
| PC-5P | 13945 | 1137 | 366 | 3651 | 3529 | 3549 | CGTTCCCGTGGTTCCCGTGG |
| 218_26568 | | | | | | | (SEQ ID NO: 2) |
| PC-5P- | 1314 | 86 | 31 | 396 | 284 | 250 | TCCACCCGTTCCCGTGG |
| 1115_3460 | | | | | | | (SEQ ID NO: 3) |
| PC-5P | 463 | 31 | 29 | 242 | 3341 | 1402 | CCCGTGGCGGTTCCCGTG |
| 445_12007 | | | | | | | (SEQ ID NO: 4) |

In addition to the 4 novel miRNA identified above, additional statistically significant miRNA biomarker panels were identified that distinguish ICH from SAH pathology and ICH-SAH-specific miRNA.

TABLE 3
Novel miRNA detected in plasma and CSF of ICH
patients and not detectable in plasma/CSF of SAH
samples. The presence of these miRNA in plasma
and/or CSF indicates ICH pathology, and not
SAH pathology.

| Reporter | Con | SAH | ICH | Con | SAH | ICH | |
|---|---|---|---|---|---|---|---|
| Name | CSF | CSF | CSF | PL | PL | PL | miR_sequence |
| PC-3P- | 0 | 0 | 5 | 0 | 0 | 96 | TTAGTATATAGGACTAACA |
| 7630_279 | | | | | | | (SEQ ID NO: 5) |
| PC-5P- | 19 | 0 | 19 | 2 | 1 | 346 | GAGGATTGGAGAGGTAGC |
| 1731_1950 | | | | | | | (SEQ ID NO: 6) |
| PC-3P- | 3 | 0 | 84 | 4 | 0 | 45 | TTGGAATCCCAGGTGTTGT |
| 4244_570 | | | | | | | TCTC (SEQ ID NO: 7) |

TABLE 4
Novem miRNA not detected in plasma or CSF of
ICH patients, but detected in SAH plasma/CSF
and control plasma/CSF samples. Absence of
these microRNA in plasma and CSF suggest
ICH pathological change.

| Reporter | Con | SAH | ICH | Con | SAH | ICH | |
|---|---|---|---|---|---|---|---|
| Name | CSF | CSF | CSF | PL | PL | PL | miR_sequence |
| PC-3P- | 147 | 103 | 0 | 58 | 63 | 0 | GTAGGTAATCTTCAGGCT |
| 3144_857 | | | | | | | (SEQ ID NO: 8) |
| PC-5P- | 66 | 55 | 0 | 78 | 45 | 0 | AGGCTAATTGATTTTGAC |
| 6497_338 | | | | | | | (SEQ ID NO: 9) |
| PC-5P- | 119 | 87 | 0 | 43 | 172 | 0 | TCTTAGGAAGCGAAGCAAT |
| 3305_806 | | | | | | | (SEQ ID NO: 10) |

### Conclusion

The results disclosed herein demonstrate that expression of these miRNA or lack thereof serve as biomarkers for ICH and SAH pathogenesis.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

1. A method, comprising:
measuring at least one of miRNA listed in Tables 1, 2, 3 and/or 4 in a cerebral spinal fluid sample and/or a plasma fluid sample obtained from a subject, wherein the at least one of miRNA comprises at least one of PC-5P-585_8337, PC-5P-218_26568, PC-5P-1115 3460 or PC-5P-445 12007.

2. The method of claim 1, wherein the at least one miRNA further comprises one or more of hsa-miR-21-5p, hsa-miR-423-5p, hsa-miR-451a_R-1, hsa-miR-338-5p_R-1, hsa-miR-16-5p, hsa-miR-204-5p, hsa-miR-100-5p_R-1, hsa-miR-151a-5p, hsa-miR-144-3p_R-1, hsa-miR-486-5p, hsa-miR-191-5p, hsa-miR-320a_R-2, hsa-miR-125a-5p_R-2m hsa-miR-126-3p_R-1, hsa-let-7b-5p, hsa-miR-186-5p, hsa-miR-125b-5p_R-2, hsa-miR-26a-5p, and hsa-miR-92b-3p, and measuring comprises measuring an increase in the expression of hsa-miR-423-5p, hsa-miR-451a_R-1, hsa-miR-338-5p_R-1, hsa-miR-16-5p, hsa-miR-204-5p, hsa-miR-100-5p_R-1, hsa-miR-151a-5p, hsa-miR-144-3p_R-1, hsa-miR-486-5p, hsa-miR-191-5p, hsa-miR-320a_R-2, hsa-miR-125a-5p_R-2, hsa-let-7b-5p, hsa-miR-186-5p, hsa-miR-125b-5p_R-2, hsa-miR-26a-5p, hsa-miR-92b-3p, PC-5p-218_26568, hsa-miR-191-5p, in the cerebral spinal fluid sample and/or a decrease in hsa-miR-21-5p, PC-5p-585_8337, PC-5p-218_26568, PC-5p-1115_3460 or PC-5p-445_12007 hsa-miR-204-5p, hsa-miR-126-3p_R-1 in the cerebral spinal fluid sample.

3. The method of claim 1, wherein the at least one miRNA further comprises one or more of hsa-miR-21-5p, hsa-miR-423-5p, hsa-miR-451a_R-1, hsa-miR-338-5p_R-1, hsa-miR-16-5p, hsa-miR-204-5p, hsa-miR-100-5p_R-1, hsa-miR-151a-5p, hsa-miR-144-3p_R-1, hsa-miR-486-5p, hsa-miR-191-5p, hsa-miR-320a_R-2, hsa-miR-125a-5p_R-2m hsa-miR-126-3p_R-1, hsa-let-7b-5p, hsa-miR-186-5p, hsa-miR-125b-5p_R-2, hsa-miR-26a-5p, and hsa-miR-92b-3p, and measuring comprises measuring an increase in the expression of hsa-miR-423-5p, hsa-miR-338-5p_R-1, hsa-miR-204-5p, hsa-miR-100-5p_R-1, hsa-miR-151a-5p, hsa-miR-486-5p, hsa-miR-191-5p, hsa-miR-125a-5p_R-2, hsa-miR-125b-5p_R-2, hsa-miR-26a-5p, PC-5p-585_8337, hsa-miR-92b-3p, and PC-5p-445_12007, or hsa-let-7b-5p, hsa-miR-125b-5p_R-2 in the plasma fluid sample and/or a decrease in hsa-miR-21-5p, hsa-miR-451a_R-1, hsa-miR-16-5p, hsa-miR-144-3p_R-1, hsa-miR-320a_R-2, hsa-miR-126-3p_R-1, hsa-let-7b-5p, hsa-miR-186-5p, PC-5p-218_26568, and PC-5p-1115_3460, hsa-miR-486-5p, in the plasma fluid sample.

4. The method of claim 1, wherein the method is a method of diagnosing a subject with subarachnoid hemorrhage (SAH) and intracerebral hemorrhage (ICH).

5. The method of claim 4, further comprising distinguishing between SAH or ICH, wherein in CFS an increase in expression of hsa-miR-204-5p relative to a control indicates SAH and a decrease in expression of hsa-miR-204-5p relative to a control indicates. ICH, and wherein in plasma an increase in expression of hsa-miR-486-5p relative to a control indicates SAH and a decrease in expression of hsa-miR-486-5p to a control indicates. ICH and a decrease in expression of hsa-let-7b-5p relative to a control indicates SAH and an increase in expression of hsa-let-7b-5p to a control indicates ICH.

6. The method of claim 1, further comprising, selecting a subject with, or believed to have, suffered a stroke.

7. The method of claim 1, further comprising, comparing the expression of one or more of hsa-miR-21-5p, hsa-miR-423-5p, hsa-miR-451a_R-1, hsa-miR-338-5p_R-1, hsa-miR-16-5p, hsa-miR-204-5p, hsa-miR-100-5p_R-1, hsa-miR-151a-5p, hsa-miR-144-3p_R-1, hsa-miR-486-5p, hsa-miR-191-5p, hsa-miR-320a_R-2, hsa-miR-125a-5p_R-2, hsa-miR-126-3p_R-1, hsa-let-7b-5p, hsa-miR-186-5p, hsa-miR-125b-5p_R-2, hsa-miR-26a-5p, PC-5p-585_8337, hsa-miR-92b-3p, PC-5p-218_26568, PC-5p-1115_3460, and PC-5p-445_12007 to a control.

8. The method of any one of claims wherein the method is used for diagnosing or prognosis of a subject with stroke.

9. The method of any one of claims 1-8, wherein the expression of miR-hsa-miR-21-5p, hsa-miR-423-5p, hsa-miR-451a_R-1, hsa-miR-338-5p_R-1, hsa-miR-16-5p, hsa-miR-204-5p, hsa-miR-100-5p_R-1, hsa-miR-151a-5p, hsa-miR-144-3p_R-1, hsa-miR-486-5p, hsa-miR-191-5p, hsa-miR-320a_R-2, hsa-miR-125a-5p_R-2, hsa-miR-126-3p_R-1, hsa-let-7b-5p, hsa-miR-186-5p, hsa-miR-125b-5p_R-2, hsa-miR-26a-5p, PC-5p-585_8337, hsa-miR-92b-3p, PC-5p-218_26568, PC-5p-1115_3460, and PC-5p-445_12007 is detected with a probe and/or primers that respectively specifically bind to hsa-miR-21-5p, hsa-miR-423-5p, hsa-miR-451a_R-1, hsa-miR-338-5p_R-1, hsa-miR-16-5p, hsa-miR-204-5p, hsa-miR-100-5p_R-1, hsa-miR-151a-5p, hsa-miR-144-3p_R-1, hsa-miR-486-5p, hsa-miR-191-5p, hsa-miR-320a_R-2, hsa-miR-125a-5p_R-2, hsa-miR-126-3p_R-1, hsa-let-7b-5p, hsa-miR-186-5p, hsa-miR-125b-5p_R-2, hsa-miR-26a-5p, PC-5p-585_8337, hsa-miR-92b-3p, PC-5p-218_26568, PC-5p-1115_3460, and PC-5p-445_12007 , or an amplification product thereof.

10. The method of claim 9, wherein the probe and/or primers are labeled with a detectable label.

11. A kit for detecting serum levels of one or more biomarkers associated with a plaque a brain hemorrhage, comprising:
a probe and/or one or more primers that specifically bind to at least one of hsa-miR-21-5p, hsa-miR-423-5p, hsa-miR-451a_R-1, hsa-miR-338-5p_R-1, hsa-miR-16-5p, hsa-miR-204-5p, hsa-miR-100-5p_R-1, hsa-miR-151a-5p, hsa-miR-144-3p_R-1, hsa-miR-486-5p, hsa-miR-191-5p, hsa-miR-320a_R-2, hsa-miR-125a-5p_R-2, hsa-miR-126-3p_R-1, hsa-let-7b-5p, hsa-miR-186-5p, hsa-miR-125b-5p_R-2, hsa-miR-26a-5p, PC-5p-585_8337, hsa-miR-92b-3p, PC-5p-218_26568, PC-5p-1115_3460, and PC-5p-445 12007.

12. The kit of claim 11, wherein the probe and/or primers are labeled with a detectable label.

13. The kit of any one of claim 11 or 12, wherein the probes are present in an array.

14. A method of treating and/or preventing subarachnoid hemorrhage (SAH) and intracerebral hemorrhage (ICH) or an associated inflammation response in a subject, comprising:
administering to the subject an effective amount of an agent that alters the expression of one or more of hsa-miR-21-5p, hsa-miR-423-5p, hsa-miR-451a_R-1, hsa-miR-338-5p_R-1, hsa-miR-16-5p, hsa-miR-204-5p, hsa-miR-100-5p_R-1, hsa-miR-151a-5p, hsa-miR-144-3p_R-1, hsa-miR-486-5p, hsa-miR-191-5p, hsa-miR-320a_R-2, hsa-miR-125a-5p_R-2, hsa-miR-126-3p_R-1, hsa-let-7b-5p, hsa-miR-186-5p, hsa-miR-125b-5p_R-2, hsa-miR-26a-5p, PC-5p-585_8337, hsa-miR-92b-3p, PC-5p-218_26568, PC-5p-1115_3460, and PC-5p-445_12007, thereby treating arterial plaque a brain hemorrhage.

15. The method of claim 14, wherein the agent decreases expression of hsa-miR-423-5p, hsa-miR-338-5p_R-1, hsa-miR-100-5p_R-1, hsa-miR-151a-5p, hsa-miR-191-5p, hsa-miR-125a-5p_R-2, hsa-miR-125b-5p_R-2, hsa-miR-26a-5p, hsa-miR-92b-3p.

16. The method of claim 15, wherein the agent is an antisense compound specific for one of hsa-miR-423-5p, hsa-miR-338-5p_R-1, hsa-miR-100-5p_R-1, hsa-miR-151a-5p, hsa-miR-191-5p, hsa-miR-125a-5p_R-2, hsa-miR-125b-5p_R-2, hsa-miR-26a-5p, hsa-miR-92b-3p.

17. The method of claim 16, wherein the antisense compound is an antisense oligonucleotide, siRNA or ribozyme.

18. The method of claim 14, further comprising administering an agent that increases the expression of at least one of hsa-miR-21-5p, hsa-miR-126-3p_R-1, PC-5p-218_26568, and PC-5p-1115_3460.

19. The method of claim 18, wherein the agent comprises one or more of hsa-miR-21-5p, hsa-miR-126-3p_R-1, PC-5p-218_26568, and PC-5p-1115_3460.

20. A composition, comprising an miRNA oligonucleotide having the nucleic acid sequence set forth as one of SEQ ID NOS: 1, 2, 3, 4, 5, 6, 7, 8, 9, or 10.

21. The composition of claim 20, further comprising a pharmaceutically acceptable carrier.

22. An nucleic acid array comprising a nucleic acid sequence that has the nucleic acid sequence set forth as one of SEQ ID NOS: 1, 2, 3, 4, 5, 6, 7, 8, 9, or 10 or complementary thereto.
